(19) United States
(12) Patent Application Publication
(10) Pub. No.: US 2017/0042480 A1
(43) Pub. Date: Feb. 16, 2017

(54) FUNCTIONAL CONTACT LENS AND RELATED SYSTEMS AND METHODS

(71) Applicant: Medella Health Inc., Kitchener (CA)

(72) Inventors: Harry Gandhi, Kitchener (CA); Huayi Gao, Kitchener (CA); Maarij Baig, Kitchener (CA); Ray Chen, Kitchener (CA)

(73) Assignee: Medella Health Inc., Kitchener, ON (CA)

(21) Appl. No.: 15/304,352

(22) PCT Filed: Apr. 15, 2015
(86) PCT No.: PCT/CA2015/000262

§ 371 Date: Oct. 14, 2016

Related U.S. Application Data
(60) Provisional application No. 61/979,887, filed on Apr. 15, 2014.

(51) Int. Cl. A61B 5/00; A61B 3/10; A61B 5/053; A61B 5/145; G02C 7/04; A61B 5/1468

(52) U.S. Cl. CPC A61B 5/6821; G02C 7/04; A61B 5/1468; A61B 5/0015; A61B 5/053; A61B 5/14546; A61B 5/742; A61B 5/1451; A61B 3/101; A61B 2562/245; A61B 2560/0214

(57) ABSTRACT

Various embodiments are described herein for a Functional Contact Lens (FCL) for detecting at least one target analyte. The FCL may comprise a substrate for supporting electronic components and providing structural support for the functional contact lens; at least one sensing element disposed on the substrate for sensing the at least one target analyte and undergoing a physical change representing a sensed signal; and an antenna disposed on the substrate for transmitting the sensed signal to an external device, the antenna being coupled to the at least one sensing element.

### FIELD

The various embodiments described herein generally relate to devices, systems and methods for functional contact lens

### BACKGROUND

A lab-on-a-chip (LOC) device may integrate one or more laboratory functions onto a single platform, which is usually a few square millimeters or centimeters in size. A LOC excels in dealing with small liquid volumes through either passive capillary forces or active pumping through various mechanisms. Therefore, a LOC closely relates to microfluidic systems in that they both manipulate miniscus amounts of samples. LOCs may also be known as Micro Total Analysis Systems (μTAS) since they not only manipulate, but also analyze sample liquids.

Due to their low fluid volume consumptions, LOCs are usually compact systems capable of massive parallelization of sample analysis. Smaller sizes also minimize fluid diffusion distances, resulting in faster analysis time, faster heat dissipation, and higher surface to volume ratios. Currently, LOCs are extensively researched and applied in the field of biomedical devices and assays, targeting major diseases such as cardiovascular disease and diabetes.

The surface of the eye is a new interface on which information may be gathered to determine the health status of patients. The tear fluid contains a variety of biomarkers whose concentrations can be correlated to biologically important health markers, such as blood composition. Therefore, detection of these biomarkers may provide a route towards non-invasive analysis of human health, with the help of a Functional Contact Lens (FCLs). Functional contact lenses (FCLs) are an emerging technology which provides a platform for the detection and analysis of biomarkers in the human tear.

### SUMMARY OF VARIOUS EMBODIMENTS

In a broad aspect, at least one embodiment described herein provides a Functional Contact Lens (FCL) for detecting at least one target analyte, comprising a substrate for supporting electronic components and providing structural support for the functional contact lens; at least one sensing element disposed on the substrate for sensing the at least one target analyte and undergoing a physical change representing a sensed signal; and an antenna disposed on the substrate for transmitting the sensed signal to an external device, the antenna being coupled to the at least one sensing element.

In at least some embodiments, the at least one sensing element may undergo an impedance change when sensing the at least one target analyte, the sensed signal causing a change in a resonance frequency or a change in amplitude of an output signal transmitted by the antenna due to the impedance change of the sensing element.

In at least some embodiments, the FCL may further comprise an impedance matching element coupled to the antenna for providing impedance matching and compensation of the antenna.

In at least some embodiments, the FCL may further comprise a second sensing element coupled to the antenna and being configured to undergo an impedance change upon sensing a second target analyte thereby generating another change in a resonance frequency or a change in amplitude of the output signal transmitted by the antenna.

In at least some embodiments, the FCL has a chip-less design.

In at least some embodiments, the FCL may further comprise a main circuit for controlling the operation of the functional contact lens; a sensing module comprising a plurality of sensing elements for sensing the at least one target analyte during use; and a modular sensor interface for coupling the main circuit to the sensing module.

In at least some embodiments, the main circuit and the modular sensor interface may be implemented using separate Application Specific Integrated Circuits (ASICs) or a common ASIC.

In at least some embodiments, the sensing elements may be electrochemical or biochemical sensors.

In at least some embodiments, the biochemical sensors may use single strain DNA based detection or antibody based detection.

In at least some embodiments, the main circuit may comprise the antenna; a communication module for receiving signals from the external device and transmitting signals to the external device; and a power module for providing power to components of the functional contact lens requiring power for operation.

In at least some embodiments, the antenna may have an annular shape and may be disposed along a first annular section of the functional contact lens having a first radius, the sensing elements may be disposed along a second annular section of the functional contact lens having a second radius less than the first radius and interconnects may be disposed along a third annular section of the functional contact lens between the first and second annular sections to couple the sensing elements with the main circuit, wherein the annular shapes, annular sections and peripheral edge of the functional contact lens may all be either circular or oval.

In at least some embodiments, interconnects may be disposed between the sensing elements to allow the sensing elements to share one or more electrodes.

In at least some embodiments, the FCL may comprise a first member having an annular shape and disposed at an outer periphery of the contact lens; a second member having a disc shape and disposed within and adjacent to the first member, and the substrate may have a disc shape with a smaller circumference than the second member and may be disposed on the second member, wherein the annular and disc shapes may all be either circular or oval.

In at least some embodiments, at least one of the first and second members may be made using a gas permeable contact lens material for users who wear the functional contact lens when sleeping.

In at least some embodiments, the at least one of the first and second members may be made using a soft hydrogel contact lens material having a high water content for users who wear the functional contact lens during daytime.

In at least some embodiments, the FCL may further comprise: an insulation layer covering at least a portion of the electronic components disposed on the substrate to provide protection; a screening layer disposed on the at least one sensing element to provide selective interaction of the at least one sensing element with its environment; and an encapsulation layer disposed over the insulation layer and the screening layer and portion of the electronic components, the encapsulation layer being configured to comprise selectively allows for diffusion of certain biomolecules towards the substrate.

In at least some embodiments, the least one sensing element may comprise a biochemical sensor and the screening layer is configured to allow for selective transmission of certain biochemicals to the biochemical sensor during use.

In at least some embodiments, the FCL may further comprise at least one optical element disposed on the substrate and the screening layer covers the at least one optical element and may be configured to allow certain target photons at certain wavelengths to pass therethrough to the at least one optical element.

In at least some embodiments, the FCL may further comprise a disintegration layer for covering at least one of at least one given sensing element and at least one given optical element, the disintegration layer being configured to disintegrate during use to allow for operation of the at least one at least one given sensing element and the at least one given optical element.

In at least some embodiments, the disintegration layer may be configured to disintegrate during use due to electrical stimulation or naturally occurring biochemically active species present in a surrounding fluid.

In at least some embodiments, the FCL may further comprise multiple similar sensing elements and sensing lifetime may be prolonged by configuring the disintegration layer to disintegrate over a subsequent sensing element after a previous operational sensing element stops functioning or is performing poorly.

In at least some embodiments, the disintegration layer may be configured for timed activation of selected sensing elements and/or selected optical elements for sequential, parallel or sequential and parallel operation thereof.

In at least some embodiments, the at least one optical element may comprise at least one of reflection pixel matrices, transmission pixel matrices, Light Emitting Diodes (LEDs) and Organic Light Emitting Diodes (OLEDs), Liquid Crystal Display (LCD), surface plasmonic resonators, and photonic crystals.

In at least some embodiments, an absorption spectrum of the at least one optical element may be electrically controlled to produce desirable wavelengths of photons that are reflected or transmitted upon incidence with the at least one optical element.

In at least some embodiments, the power module may comprise: a rectifier for converting harvested energy conditioning the stored energy to provide power; an energy storage unit for storing converted harvested energy for use by electronic components of the functional contact lens; and at least one energy harvesting element comprising at least one of one or more fuel cells, one or more solar cells and one or more piezoelectric cells.

In at least some embodiments, the one or more piezoelectric cells may comprise one of micro-pillars and nano-pillars that create a piezoelectric potential upon mechanical deformation.

In at least some embodiments, the at least one sensing element may be coupled with a sensor interface for receiving an input signal and providing an output signal, and the at least one sensing element may comprise: a working electrode having an annular shape; and a second electrode having an annular shape with a larger radius that surrounds a majority of the working electrode, the second electrode being configured to operate as a counter electrode or a reference electrode.

In at least some embodiments, the at least one sensing element may be coupled with a sensor interface for receiving an input signal and providing an output signal, and the at least one sensing element may comprise: a working electrode having an annular shape; a counter electrode having an annular shape that surrounds a majority of the working electrode; and a reference electrode having an annular shape that surrounds the counter electrode.

In at least some embodiments, the reference electrode may comprise two electrodes having semi-annual shapes disposed on either side of the counter electrode.

In at least some embodiments, the at least one sensing element may comprise a fourth electrode disposed within the annular shape of the working electrode, the fourth electrode being configured to provide at least one of a modulating function and a cleansing function of a microenvironment of the working electrode.

In at least some embodiments, the at least one sensing element may comprise a modulating electrode and a cleansing electrode that are both disposed within the annular shape of the working electrode and being configured to provide a modulating function and a cleansing function, respectively, of a microenvironment of the working electrode.

In at least some embodiments, the working electrode may be interdigitated.

In at least some embodiments, the input signal and the output signal may be one of a constant DC voltage, a constant DC current, a step-up DC voltage, a step-up DC current, a sinusoidal AC voltage with a certain radial frequency and amplitude, a sinusoidal AC current with a certain radial frequency and amplitude, a square wave AC voltage or current pulse, or any combination thereof.

In at least some embodiments, the at least one target analyte may comprise at least one of acids, ions, carbohydrate, proteins, enzymes, lipids, antigens, hormones, nucleic acids, small molecules, medications and recreational drugs.

In at least some embodiments, the substrate may have an annular or wedge shape.

In a broad aspect, at least one embodiment described herein provides a system for monitoring a person's health, wherein the system may comprise: a Functional Contact Lens (FCL) that monitors at least one condition for the person; a transceiver-reader device that communicates with the FCL; and an external processing device, wherein the transceiver-reader device acts as a relay device in sending signals between the FCL and the external processing device.

In at least some embodiments, the FCL may be defined in accordance with any of the embodiments described in accordance with the teachings herein.

In at least some embodiments, the communication between the FCL and the transceiver-reader device may be bi-directional.

In at least some embodiments, the FCL may directly communicate with the external reader device and the communication is bi-directional.

In at least some embodiments, at least one of the transceiver-reader device and the external reader device may be configured to provide RF power signals to the FCL to wirelessly power the FCL.

In at least some embodiments, at least one of the transceiver-reader device and the external processing device may process and/or display received data from the FCL.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described.

FIG. 1 is a top view of an example embodiment of an annular Functional Contact Lens (FCL).

FIG. 2 is a top view of an example embodiment of an annular “chip-less” FCL.

FIG. 3 is a top view of an example embodiment of a wedge shaped “chip-less” FCL that may be placed in the conjunctive sac of the user's eye.

FIG. 4 is a top view of an example embodiment of a wedge shaped FCL that may be placed in the conjunctive sac of the user's eye.

FIG. 5 is a magnified cross-sectional view of an example embodiment of an FCL.

FIG. 6A is a schematic view of an example embodiment showing a sensor interface circuit and a sensor element along with an input and an output for measurements.

FIG. 6B is a schematic view of an example alternative embodiment showing a sensor interface circuit and a sensor element along with an input and an output for measurements.

FIG. 6C is a schematic view of another example alternative embodiment showing a sensor interface circuit and a sensor element along with an input and an output for measurements.

FIG. 7 is a block diagram showing an example embodiment of a telemetry system involving an FCL

FIG. 8 is a block diagram showing another example embodiment of a telemetry system involving an FCL.

FIG. 9 shows an example of flow of power and information among components of an example FCL including sensing modules, transceiver-reader device, and external processing device.

FIG. 10 shows an example of the flow of information and power among components of an example FCL including optical modules, external transceiver-reader device, and external processing device.

Further aspects and features of the embodiments described herein will appear from the following description taken together with the accompanying drawings.

### DETAILED DESCRIPTION OF THE EMBODIMENTS

Various apparatuses or processes will be described below to provide an example of at least one embodiment of the claimed subject matter. No embodiment described below limits any claimed subject matter and any claimed subject matter may cover processes, apparatuses or systems that differ from those described below. The claimed subject matter is not limited to apparatuses, processes or systems having all of the features of any one apparatus, process or system described below or to features common to multiple or all of the apparatuses, or processes or systems described below. It is possible that an apparatus, process or system described below is not an embodiment of any claimed subject matter. Any subject matter that is disclosed in an apparatus, process or system described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical element or electrical signal (either wired or wireless) or a mechanical element depending on the particular context.

It should be noted that terms of degree such as “substantially”, “about” and “approximately” as used herein mean a reasonable amount of deviation of the modified term such that the end result is not significantly changed. These terms of degree may be construed as including a certain deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Various embodiments for a Functional Contact Lens (FCL) and related systems and methods are provided according to the teachings herein. The various FCLs described herein may be considered to be lab-on-chip (LOC) systems which may generally manipulate and detect at least one target biomarker in interstitial fluids, such as the human tear. An encapsulation contact lens material may be used for structural integrity of the FCL. Together the LOC and the encapsulation material make up the FCL platform.

At least some FCL embodiments described herein may use one or more functional hydrogel layers for the manipulation of molecules, and one or more sensors for detection of target molecules. Information related to the target molecules may then gathered by a processing device on the FCL and transmitted by a communication module on the FCL having antennas to an external processing device for further analysis.

In some embodiments, a user health profile may be generated by correlating concentrations of a target biomarker in different interstitial fluid and/or blood, or multiple target biomarkers in the same fluid. For example, glucose, calcium, sodium, ascorbic acid, uric acid, lysozyme, or IGE may provide a correlation between tear and blood biomarkers. Accordingly, the FCL may be used to non-invasively monitor and provide comprehensive biomedical information about the user from various biomarkers in the user's tear fluid in a periodic, continuous or other timed manner.

In some alternative embodiments, the FCL may include optical elements for optical information to be processed and displayed to the user or to external photo-detecting devices.

To be directly in contact with a user's interstitial fluid such as the basal tear, for example, the FCL may be placed onto the eye, either on top of the cornea or in the conjunctive sac. Target biomarkers, after diffusing through various layers of the FCL, may come in contact and react with sensing module elements of the FCL, which will generate electrical signals. These signals may be processed by processing elements in the FCL, such as integrated circuits (ICs), and the processed signals or other related information may be transmitted by antenna situated in the FCL to an external transceiver-reader.

The external transceiver-reader device may receive and store information sent from the FLC and/or relay the information to an external processing device such as, but not limited to, a cellphone, a computer, an infusion pump, or any other suitable type of smart electronic devices. Information may then be processed further and stored in secure locations.

In some embodiments, the FCL may directly communicate with the external processing device, provided the external processing device can receive and process the transmitted information and, in some embodiments, provide power to the FCL wirelessly. One example of such a device is a smart phone with a near field communication (NFC) antenna and battery. The smart phone will be able to wirelessly transmit power to the FCL via inductive coupling at NFC frequencies, and at the same time, receiver biomarker profile information from the FCL.

Alternatively, the FCL may incorporate energy harvesting units such as fuel cells, solar cells, or electromechanical cells such as piezoelectric cells. The energy generated by the energy harvesting unit may be stored in one of a capacitor, a super-capacitor, or a battery, and used to power other electronic components in the FCL system.

In some embodiments, an IC on the FCL may not be needed for the external transceiver-reader device to monitor the biomarker profile in the interstitial fluid. In this case, the sensing module may be incorporated into the antenna, such that a change in resonant frequency or amplitude of a transmitted wireless signal may be detected upon impedance change of the sensing module due to varying biomarker concentrations.

In general, the various LOC system embodiments described herein incorporate multiple functional layers, including various insulation, screening, and disintegration layers, as well as electronic components, including a sensing module, an optical module (which is optional), a communication module, a power module and an antenna. An IC situated on the FCL may include one, many or all of the electronic modules mentioned above.

The Insulation layer may be used to prevent water and reactive species from damaging the electronic components of the FCL. The screening layers may provide at least one of chemical and optical screening functions, improves the performance of the sensing and optical modules by stopping undesirable biochemical species and/or unwanted ambient photons from reaching the sensing or optical components of the FCL. For example, the screening layer may be used to screen different target analytes by letting them pass through the screening layer and/or for screening certain target photons at certain wavelengths by letting them pass through the screening layer. The disintegration layer may be used to encapsulate the sensing and/or optical modules and disintegrate with time or active electric stimulation, such as a current, so that functional modules encapsulated by the disintegration layer may be activated after prolonged periods of time.

In some embodiments, multiple ICs may be incorporated and connected via interconnects for the purpose of modular design.

In some embodiments, individual system components may be connected via interconnects on the functional contact lens.

The various embodiments of LOC systems described in accordance with the teachings herein may be used with contact lenses of different shapes, such as annular and wedge-shaped designs, for example.

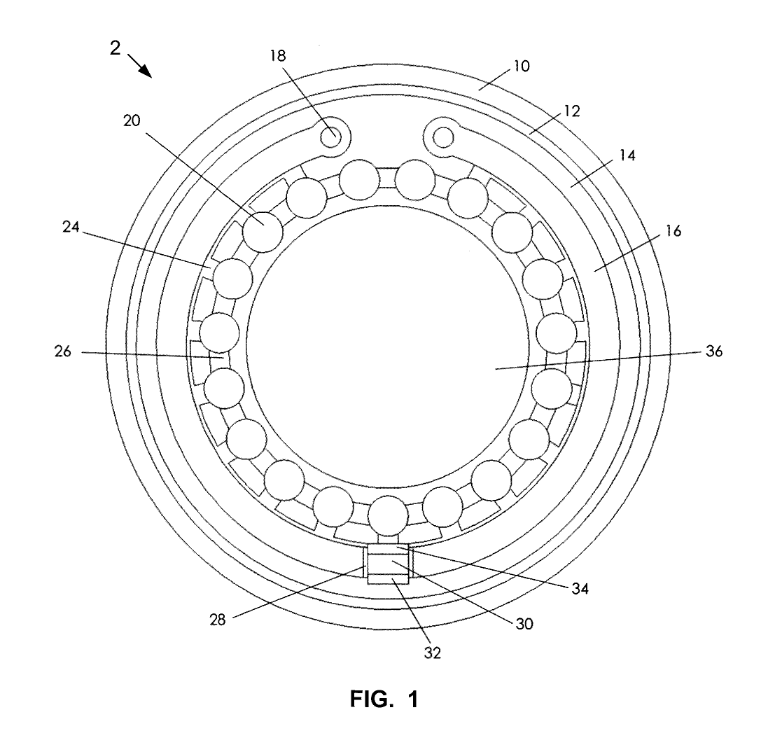

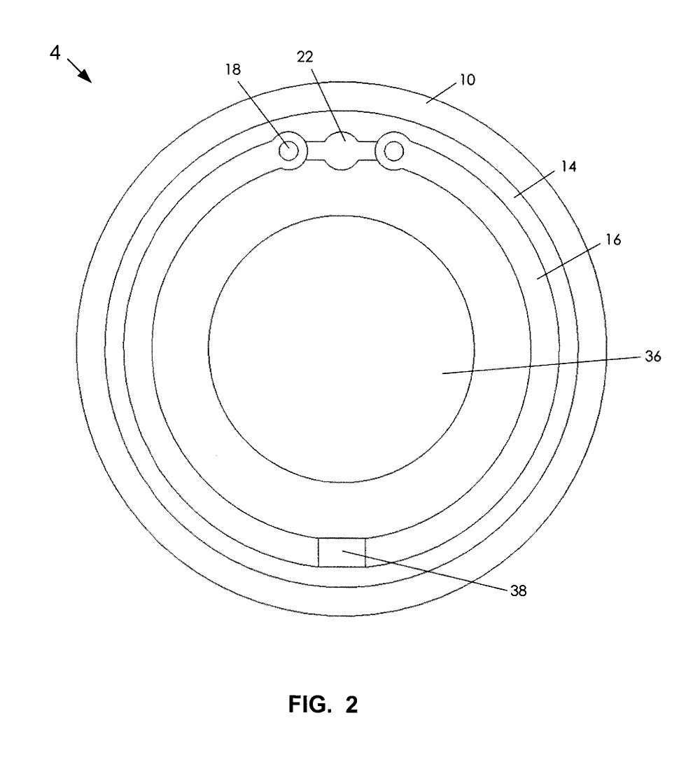

An annular contact lens design (see FIGS. 1-2) is one where the contact lens has either vision correction functions or no vision correction functions while carrying the LOC system. In this design, the LOC system resides on the periphery or outer annular regions of the contact lens to have minimal optical interference with the sight of the contact lens wearer.

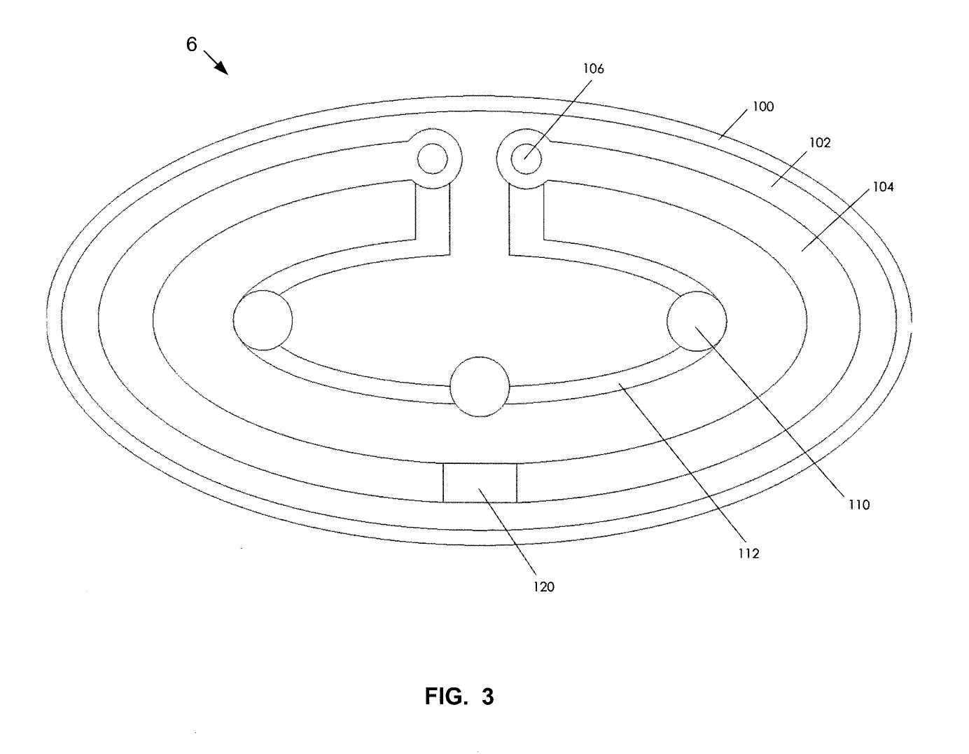

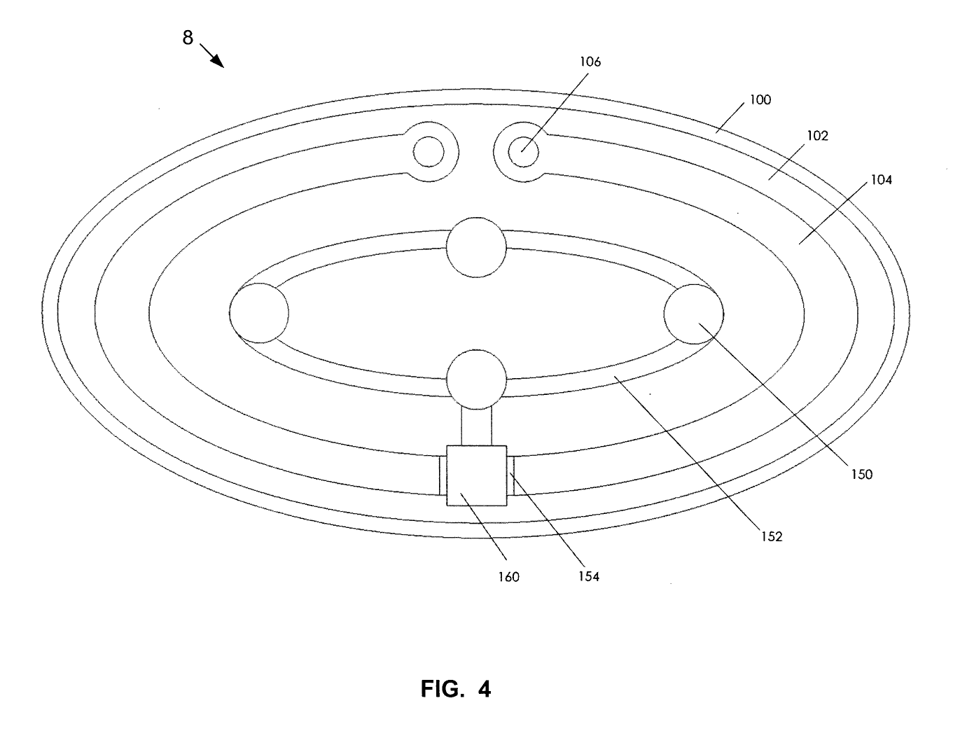

A wedge contact lens design (see FIGS. 3-4), is shaped so that it may be placed within the conjunctive sac. In these embodiments, the LOC system may cover the entire area of the lens since there is no interference with the user's sight and a vision correction mechanism may not be used. However the wedge-shape provides additional space for system components to reside.

In the various embodiments described in accordance with the teachings herein, the thickness of the FCL may be about 300 micro-meters, in order to reside inside the basal tear film of the user's eye for comfort.

In at least some embodiments described in accordance with the teachings herein, the periphery of the FCL may be made of soft hydrogel material to maximize comfort for the user.

In at least some embodiments described in accordance with the teachings herein, different areas of the FCL may be made of different materials. For example, in some embodiments, the FCL may comprise an annular center piece made of silicon elastomer, and an outer ring piece made of hydrogel materials. Alternatively, in some embodiments, the FCL may comprise an annular center piece made of hydrogel materials, and an outer ring piece made of silicon elastomer.

Referring now to FIG. 1, shown therein is a top view of an example embodiment of an annular FCL 2. The FCL 2 comprises a first member 10, a second member 12, and a substrate 14 that may be used to provide support for the components of the LOC, which in this case includes an antenna 16, sensing structures 20, 24 and 26, interconnects 28 and processing units 30, 32 and 34. The region 36 is free from components and provides a visual pathway for the user's eye so that the user can see the surrounding environment.

The first member 10 is an outer ring or annulus that extends along the outer periphery of the FCL 2 and the second member 12 has a disc-shape that is encircled and touches the first member 10. Together the first and second members 10 and 12 provide a bottom portion of the housing for the FCL 2. The boundary between the first and second members 10 and 12 may be determined based on desired comfort levels, usage cases, and properties of the user's eye rather than the location of the electronic components of the LOC.

The first and second members 10 and 12 may be made from the same or different materials, based on a desired usage of the FCL 2. For example, for users who wear the FCL during sleep, at least one of the first and second members 10 and 12 may be made from gas permeable contact lens materials such as, but not limited to, silicon elastomers, for example. For daytime wear, at least one of the first and second members 10 and 12 may be made using a soft hydrogel contact lens material having a high water content, for example.

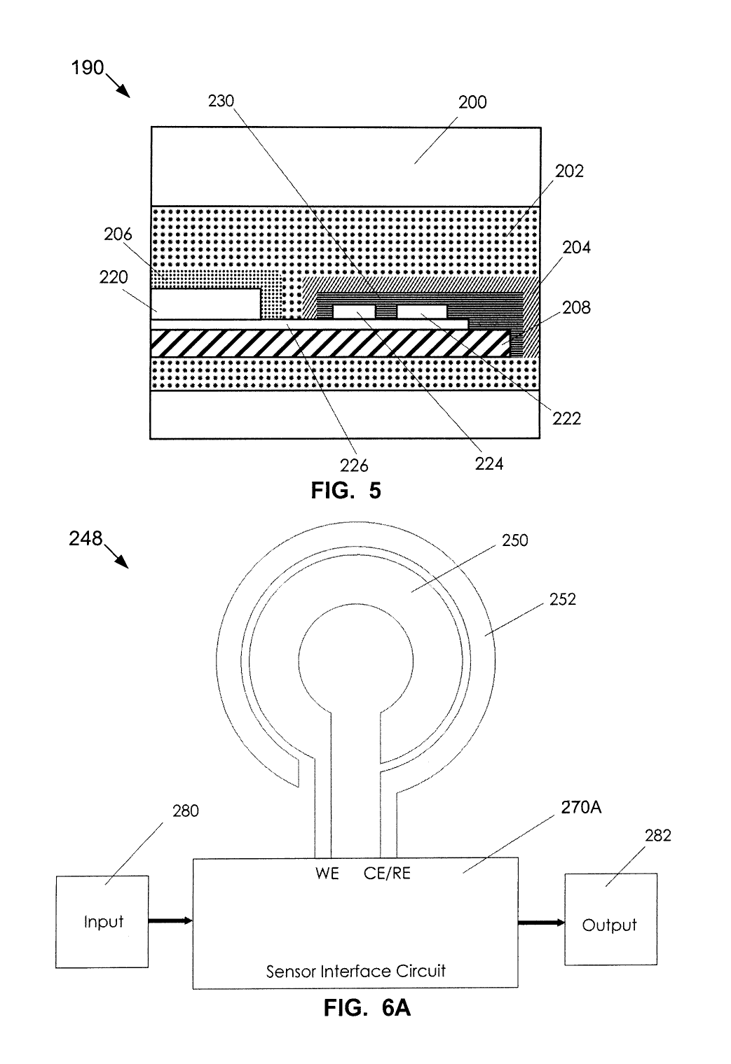

The substrate 14 is another ring having a 3D volume that is disposed on top of the second member 12. The substrate 14 supports the electronic components 16, 18, 20, 24, 26, 28, 30, 32 and 34 of the FLC 2. The substrate 14 may also comprise various functional layers such as a disintegration layer (optional), a screening layer, and an insulation layer. For example, in some embodiments, the substrate 14 may include (from the top to the bottom): an encapsulation layer, a disintegration layer (optional), a screening layer, an insulating layer, electronic components including sensing elements, optical elements (optional), a substrate material, and another encapsulation layer. An example showing the layout of these layers is shown in FIG. 5.

The antenna 16 may comprise one or more loops. When the antenna 16 is multi-layered, then vias or through-holes 18 may be used to vertically physically couple portions of the antenna 16 that are on different layers of the substrate 14. Depending on the number of layers that are used for the antenna 16, different antenna designs may incorporate different locations, numbers and sizes of the through-holes. Examples of multi-layer antennas are shown in U.S. provisional patent application No. 62/066,805 filed on Oct. 21, 2014, which is hereby incorporated by reference in its entirety. In some cases, the antenna may be disposed entirely on one surface and vias or through-holes are not used.

The sensing structure 20 is an example of a biosensor which may be an artificial enzymatic biosensor or an artificial non-enzymatic biosensor as described in U.S. provisional patent application No. 62/115,886 filed on Feb. 13, 2015, which is hereby incorporated by reference in its entirety. Other types of sensors that may be used include label-free electrochemical immunoassay, for example. The example embodiment shows a plurality of biosensors but only one of them is labelled for ease of illustration. It should be noted that there may be from 1 to N biosensors 20 in a given embodiment of the FCL 2, where N is an integer larger than 1.

A sensor module (i.e. biosensor module) for the FCL 2 includes all of the sensors 20 (i.e. biosensors 20) along with multiple electrodes that facilitate electrochemical reactions with various desired target species. The overall shape of the biosensor module may be, but is not limited to, annular, polygonal or fractal, for example. The shape of the biosensor module may be determined based on a desired surface area that will be used as active detection sites for the biosensors 20. In some embodiments, the biosensors 20 may reside on multiple vertical layers of the substrate 14.

The biosensor module also includes a plurality of interconnects 24, only one of which is labelled for simplicity. The interconnect 24 may be used to physically and electrically couple the processing units 30, 32 and 34 with the biosensor module. In some embodiments, interconnects 24 may also be used to house the working electrodes for each biosensor.

The biosensor module may also include a plurality of interconnects 26, only one of which is labelled for ease of illustration, for physically and electrically connecting two or more biosensors together. Interconnects 26 may be used to house at least one of counter electrodes, reference electrodes, modulating electrodes, and cleansing electrodes, which may be shared amongst two or more biosensors 20.

Interconnects 28, only one of which is labelled for ease of illustration, may be used to couple the processing units 30, 32 and 34 with the antenna 18.

The processing units 30, 32 and 34 may be integrated circuits or other suitable micro or nano electronics. The processing unit 30 may comprise various electronic components including one, many, or all of a communication module (not shown), a power module (not shown), and the interface circuits that provide interfaces between sensing and/or optical modules, the communication module and the power module. The processing unit 34 may comprise a separate sensor interface integrated circuit, which may contain the interface circuit for one, several or all of the biosensors in the biosensing module. The processing unit 32 may be a separate power integrated circuit that may be used to couple with the power module (not shown). The power integrated circuit may comprise energy storage units such as at least one capacitor, at least one super-capacitor, at least one battery cell, power electronics, and/or one or more energy harvesting elements such as at least one fuel cell, at least one solar cell, at least one piezoelectric cell or a combination thereof.

In an alternative embodiment of the FCL 2, at least one of the biosensors 20 and/or region 36 may be part of an optical module that comprises reflection and/or transmission pixel matrices. A reflection pixel matrix contains a mirror layer which reflects external photons having certain wavelengths that were absorbed by the pixel matrix. The reflected light may be observed by external optical elements, such as a photo-detector or human eyes. A transmission pixel matrix has transparent conducting layers, so that incident photons at certain wavelengths that were absorbed by the pixel matrix may be transmitted. The transmitted photons may be observed by the user's eyes.

In some embodiments, the biosensors used by any FCL described herein may include functional hydrogel layers having micro-hydrogel particles that may qualitatively detect the presence of target analytes. These types of biosensors may be functionalized with single-strain DNAs or specific antibodies coded with fluorophores so that upon target analyte binding with the biosensor, a fluorescent signal may be generated and outputted.

In some embodiments, the sensors used in any FCL described herein may monitor the local environment of the FCL continuously, periodically or intermittently by measuring certain physical attributes such as temperature, ocular pressure and pH.

Target biomarkers or target analytes that may be sensed by the biosensors used in the FCLs described herein may be molecules that are within the precorneal tear, such as acids, ions, carbonhydrate, proteins, enzymes, lipids, antigens, hormones, nucleic acids, small molecules, medications and recreational drugs, for example. Acids and their conjugate bases of interest may include ascorbic acid/ascorbate carbonic acid/carbonate, lactic acid/lactate, pyruvic acid/pyruvate and uric acid/urate, for example. Ions of interest include calcium, potassium, sodium, and magnesium, for example. Carbonhydrates of interest may include fructose, glucose, sucrose, glactose, maltose, and lactose, for example. Proteins of interests may include lysozyme, lipocalin, tear-specific pre-albumin (TSP), cytokine (tumor necrosis factors, TNF), lipocalin, epidermal growth factor (EGF), insulin-like growth factor (IGF-1, IGP-BP-3), albumin, antiproteinases, interleukins (1 family, 2, 4, 6, 7, 10), secretory component (SC), glycoproteins (alpha-1-anti-chymotrypsins, fibrinogen), orosomucoid, transferrin (lactoferrin) and ceruloplasmin, ferritin, procalcitonin (PCT), C-reactive protein (CRP) for example. Enzymes of interest may include hexokinase, aldolase, triose phosphate isomerase, phosphoglucoseisomerase, pyruvate kinase, enolase, lactic dehydrogenase (five isoenzymes), citrate synthase, aconitase, phosphofructokinase, glyceraldehyde-3-phosphate dehydrogenase, phosphoglyceratemutase, pyruvate dehydrogenase, isocitric dehydrogenase, α-ketoglutarate dehydrogenase, succinyl-Coa-synthase, succinate dehydrogenase, fumarase, matalate dehydrogenase, glucose 6-phosphate dehydrogenase, 6-phosphogluconolacotne, 6-phosphogluconate dehydrogenase, ribulose-5-phosphate isomerase, transketolase, transaldolase, transketolase, lysozyme, amylase, proteases, antiproteases, peroxidase, plasminogen activator and lysosomal acid hydrolases, for example. Lipids of interest may include wax esters, sterol esters (mainly cholesterol), polar lipids, hydrocarbons, diesters, triglycerides, free sterols and free fatty acids, for example. Antigens of interest may include those belonging to adenoviruses, staphylococcus bacteria, streptococcus bacteria, Hemophilus influenza bacteria, Chlamydia, and gonorrhea, PSA, CEA, for example. Hormones of interest may include cortisol, catecholamines, endorphins, insulin, dehydroepiandrosterone (DHEA), thyroid stimulating hormones (TSH), thyroxine (T4), epinephrine (EPI), norepinephrine (NE), and dopamine, for example. Nucleic acids of interest may include DNase (Deoxyribonuclease I, II, III), RNase (Ribonuclease A, H, I, II, III, D, PhyM, R, T, TI, T2, U2, V1, V,) Exorinounuclease I, II and recombinant DNase/RNase. Small molecules of interest may include urea and ethanol, for example. Medications of interest may include ibuprofen, acetaminophen, alrex, betaxon, besivance, cosopt, lucentis, metformin, sulfonylureas, meglitinides, thiazolidinediones, Adriamycin, adruicil, Cytoxan, ethyol, and leukeran, for example. Recreational drugs of interest may include psychedelics, opium, LSD, barbiturates, benzodiazepines, amphetamines, ecstasy (MDMA), cocaine, heroin, cannabis, for example.

Referring now to FIG. 2, shown therein is a top view of an example embodiment of an annular “chip-less” FCL 4. In this example embodiment, the FCL 4 does not have any integrated circuits or devices for processing, sensing, communication, or power. Instead, the sensing element 22 is coupled with the antenna 16. The sensing element 22 is a one electrode system that uses a Working Electrode (WE). The rest of the elements for FCL 2 function similarly and are implemented as was described for FCL 2.

The presence of a target biomarker to which the sensing element 22 will react will change the impedance of the sensing element 22, which in turn changes the overall impedance of the antenna 16. This change in impedance may result in a change of resonant frequency or amplitude for a signal that is transmitted by the antenna 16. The change in this transmission signal may be detected by an external transceiver-reader device worn by the user or an external processing device such as, but not limited to, a cellphone, a computer, an infusion pump, or any smart electronic device. In this embodiment an impedance matching element 38 is used for impedance matching and compensation of the antenna 16. The impedance matching element 38 may be a passive electronic component such as, but not limited to distributed conductive traces, solid state capacitors, inductors, resistors or combinations thereof, for example.

In an alternative embodiment, element 38 may instead be another sensing element which detects a different biomarker from that detected by sensing element 22. Since element 38 is a part of the antenna 16, the sensing mechanism that is used may be the same as element 22. However, the functional layer can vary, enabling sensing of different biomarker.

In another alternative embodiment, elements 22 and 38 may be energy harvesting units such as, but not limited to, a fuel cell, a solar cell, or an electromechanical cell which generates voltage or current. The energy harvesting units may provide unregulated voltage/current to the sensing element if it is required. One example of a piezoelectric cell is Zinc Oxide micro/nano pillars, which create a piezoelectric potential upon mechanical deformation (such as due to blinking). This piezoelectric potential may aid the sensing element, or other cells, such as a fuel cell for example, to react with a target analyte in a different way compared to a sensing analyte such as for generating current upon detection of a target analyte. Solar cells may also act as current source under different voltage conditions in which a similar mechanism may apply. The energy harvesting units may include a combination of the different types of energy harvesting elements described herein for generating energy in different sensing applications.

Referring now to FIG. 3, shown therein is a top view of an example embodiment of a wedge shaped “chip-less” FCL 6 that may be placed in the conjunctive sac of the user's eye. The FCL 6 does not include any integrated circuits. The FCL 6 comprises a first member 100, a substrate 102 that may be used to provide support for the components of the LOC, which in this case includes an antenna 104, sensing elements 110, and interconnects 112 and impedance matching elements 120. The horse-shoe shaped region between the antenna 104 and the sensing elements 110 are interconnects 112.

The first member 100 is made of contact lens material, and has an oval or oblong 3D disc shape that extends along the entire bottom surface of the FCL 6. The first member 100 may be made from gas permeable silicon elastomers, hydrogels, or a combination thereof.

The substrate 102 may be comprised of various functional layers such as a disintegration layer, a screening layer, and an insulation layer (all not shown). The disintegration layer may be optional, if timed activation of sensing and/or optical elements is not needed.

The antenna 104 comprises vias or through-holes 106 when the antenna 104 is a multi-layered antenna. The vias 106 physically couple loops from different layers of the substrate 104 as described previously for the FCL 2.

The sensing element 110, only one of which is labelled for ease of illustration, comprises a single electrode which contains the W.E. which may be non-catalytic and a functional layer which changes impedance upon binding to a specific target analyte. Interconnects 112, of which only one is labelled for ease of illustration, electrically and physically couple the sensing elements 110 to one another as well as to the antenna 104 for quality assurance and multiple analyte sensing capabilities.

The W.E. may provide the ideal conditions for fabrication of a functional layer. Additionally, the W.E. may connect the functional layer with the antenna 104 such that a change in impedance of the functional layer is relayed to the antenna 104. The W.E. may be fabricated using clean room, chemical, electrochemical, or screen printing techniques.

In some cases a multi-impedance sensor design (e.g., see FIG. 3) is useful for increasing the quality of the signal coming from the primary sensor. This may be done by placing other sensors in the circuit that react to an increase in a certain analyte by changing the impedance out of the normal range measured by the primary sensor. The concentration of analytes measured by the quality assurance sensors may normally be at a constant low value.

In other embodiments, the signal from each sensor may be differentiated by altering the frequency range, functional material, and altering the sensing environment for each sensor.

Since the FCL 6 is covered by the conjunctive sac (i.e. lower eye lid of the user), the components locations depend on performance and comfort. For example, the IC may be situated in the very center of the FCL, where thickness is highest, which cannot be done if the FCL has an annular shape and is on the cornea. Another example may be to use a multi-layered antenna, increasing the thickness of the FCL but decreasing the radii for a smaller footprint.

Referring now to FIG. 4, shown therein is a top view of an example embodiment of a wedge shaped FCL 8 that may be placed in the conjunctive sac of the user's eye. The FCL 8 comprises a first member 100, a substrate 102, an antenna 104, sensing elements 150, interconnects 152 and 154, and a processing device 160. The components for the FCL 8 are implemented similarly and are functionally similar to corresponding elements in the FCL 6. There is no visual pathway in the middle of the FCL since the user won't be looking seeing through the FCL. As a result, there is more freedom to arrange the components in the encapsulation material, and the overall FCL may be thicker. Again, since the FCL 8 is covered by the conjunctive sac (i.e. lower eye lid of the user), the components locations depend on performance and comfort.

Referring now to FIG. 5, shown therein is a magnified cross-sectional view of an example embodiment of an FCL 190 in its environment which is a bulk analyte fluid 200 having upper and lower layers on either side of the FLC 190. In this example embodiment, the FCL 190 comprises encapsulating contact lens layers 202, a substrate 208, a multipurpose antenna 226, an integrated circuit (IC) element 220, a sensing element 222, an optical element 224, an insulating layer 206, a screening layer for biochemicals and/or optics 230, and a disintegration layer 204. Some of these elements may be optional and may not be used in other embodiments. For example, in some cases, timed activation of the sensing elements 222 and the optical elements 224 is not used so the disintegration layer is not needed.

In the example embodiment of FCL 190 shown in FIG. 5, the encapsulating contact lens layers 202 are on both surfaces of the FCL 190 that face towards and away, respectively, from the user's eye. The substrate 208 may be disposed adjacent to a portion of the surface of the FLC 190 that faces the user's eye in some embodiments and in other embodiments this orientation may be flipped. The multipurpose antenna 226 is then deposed on a portion of a surface of the substrate 208 that is opposite the surface of the substrate 208 that is adjacent to the encapsulating contact lens layer 202. Other electrical components are deposed on other regions of the substrate 208 depending on the particular functionality and the layout design of the electronic components of the FCL 190. In this example embodiment, the electronic components include the IC 220, the sensing element 222, and the optical element 224. It should be noted that in other embodiments there may be two or more sensing elements 222 and/or two or more optical elements 224. The insulating layer 206 surrounds the IC 220 to protect it from fluids. The screening layer 230 to screen for particular biochemicals and/or optics 230 surrounds the one or more sensing elements 22 and the one or more optical elements 224. The disintegration layer 204 surrounds the screening layer 230 and during use the disintegration layer 204 may disintegrate or be removed due to the presence of certain components in the bulk fluid 200 which may represent certain biological conditions or events.

The outer surface of the FCL 190, e.g. the surface of the FCL 190 that faces away from the user's eye, has access to the bulk analyte fluid 200. The bulk analyte fluid is a fluid that contains target analytes of interest that are to be detected or measured by the FCL 190. In general, the bulk analyte fluid 200 may include interstitial fluid, plasma, blood, sweat, urine, and saliva. However, for applications in which the FCL 190 is worn by a user, the bulk fluid comprises interstitial fluids such as basal tear fluids which form a tear film on the surface of the eye.

The tear film is a complex functional layer which protects the eye from infection and provides nutrition to the cornea. The tear film is about 7 um thick and consists of three layers: i) a thin mucin layer ii) a thick middle aqueous layer and iii) a thin lipid surface layer [1]. Tears have been shown to contain lipids, mucins, ions, proteases, immunoglobulins, catecholamines, endorphins, and multiple other small molecules. The close compositional similarity (see Table 1) between blood and tears enables detection and management of multiple medical conditions (see Table 2).

TABLE 1
Common species in tear and blood (see references in App. A)

| Biomarker | Tear | | Blood | |
|---|---|---|---|---|
| Na⁺ | 120-165 | mM | 130-145 | mM |
| K⁺ | 20-42 | mM | 3.5-5 | mM |
| Ca²⁺ | 0.4-1.1 | mM | 2.0-2.6 | mM |
| Mg²⁺ | 0.5-0.9 | mM | 0.7-1.1 | mM |
| Cl⁻ | 118-135 | mM | 95-125 | mM |
| HCO³⁻ | 20-26 | mM | 24-30 | mM |
| Glucose | 0.1-0.6 | mM | 4-6 | mM |
| Urea | 3.0-6.0 | mM | 3.3-6.5 | mM |
| Lactate | 2-5 | mM | 0.5-0.8 | mM |
| Pyruvate | 0.05-0.35 | mM | 0.1-0.2 | mM |
| Ascorbate | 0.008-0.04 | mM | 0.04-0.06 | mM |
| Total protein | ~7 | g/L | ~70 | g/L |

The encapsulating contact lens layer 202 may be made from hydrogels, silicon hydrogels, or silicon elastomer or any combination of two or more of these materials where one of these materials may form the core of the layer 202 while the other materials may form the external portions of the encapsulation lens layer 202. For example, the encapsulating contact lens layer 202 may comprise a selective, nano-porous hydrogel structure that selectively allows for diffusion of biomolecules according to at least one of lipophilicity, molecular weight, charge, and degree of ionization. The nano-porous hydrogel structure may also disallow the passage of unwanted molecules, where molecule fouling on the surface of the FCL is cleared with the natural blinking motion of the eye. The passage selection structure allows for selected molecules to reach a corresponding sensor which leads to an increase in the signal to noise ratio. In some embodiments, this passive selection structure may also provide higher target molecule diffusion rates for faster response time.

TABLE 2
Common biomarkers in tear and blood (see references in App. A)

| Biomarker | Tear | | Blood | | Conditions |
|---|---|---|---|---|---|
| IL-6 | 30-100 | pg/mL | 1.56-8.6 | pg/mL | Inflammation |
| IL-8 | 1330-2000 | pg/mL | 3.9-76.0 | pg/mL | Inflammation |
| A1ACT | 11.0-21.0 | mg/mL | 0.16-0.18 | μg/mL | Alzheimer, MS |
| Choles- | 0.1-1.0 | mM | 3-7 | mM | Cardiovascular |
| terol | | | | | |
| Cortisol | 9.8 | ng/mL | 210.4 | ng/mL | Hypocortisolism |
| DHEA | 0.581 | ng/mL | 8.2 | ng/mL | Hyperthyroidism |
| Lactic | 1-5 | mM | 0.5-2 | mM | Metabolism |
| Acid | | | | | |

Hydrogels have lower oxygen transmission, in addition to lower water content. This minimizes swelling when exposed to a solution and thus stress and strain forces are minimized throughout the material. Fragile electronic, optical or sensing components can be expected to experience minimal perturbation in the form of stress and strain when the encapsulating hydrogel material swells in water. Hydrogels are also very hydrophilic and thus provide greater physical comfort for a contact lens wearer. However, hypoxic complications may arise on the cornea due to low oxygen transmissibility of hydrogels. As thickness increases for the hydrogel substrate, the hypoxia effect also increases. Therefore, it may be desirable to encapsulate the electronics in a very thin hydrogel layer, as swelling is minimized while oxygen transmission is maximized.

In some cases, silicon based hydrogels (SiHy) and silicone elastomers can be used to increase oxygen transmission, thereby preventing hypoxia. The incorporation of siloxane groups may also increase the modulus and stiffness of SiHys, producing a more physically robust contact lens. However, SiHys and silicone elastomers are generally more hydrophobic than hydrogels and may result in wettability issues for some contact lens wearers. Water content is also higher in SiHys and in silicone elastomers as compared to hydrogel, which results in greater stress and strain forces during the expansion process that can affect the embedded electronics.

The disintegration layer 204 which covers the sensing and optical elements 222 and 224 may be disintegrated by electrical stimulation such as current, or by naturally occurring biochemically active species such as enzymes that are present in the surrounding fluid. In the first mechanism, the Electro-Stimulated Erosion Mechanism (ESEM), erosion of an encapsulating gel in response to an electric stimulus can be utilized for selective exposure of a sensing element to the fluid environment of the FCL 190. The gel used in the disintegration layer 204 may be made using two or more oppositely-charged water-soluble polymers that form hydrogen bonding interactions in a certain pH range. Electrical stimulation results in electrolysis of water which increases the local pH at the surface of the two polymers, thereby disrupting the hydrogen bonding interactions and causing disintegration. In the second mechanism, the Enzyme-related Erosion Mechanism (EEM) may be applied. The enzyme resilient polymer is synthesized using the enzyme reactant as the monomer. As the enzyme concentration increases locally, the polymer is acted upon, resulting in erosion of the polymer.

When considering the device lifetime and replacement frequency, normally, the limiting factor may be the lifetime of the sensing elements. However, by using an electrically stimulated gel erosion process, this lifetime may be increased. In some embodiments, an array of erosion encapsulated sensors may be created initially, and since dry shelf life is long, a new sensor element can be uncovered at a defined point in time or when the sensitivity value of a similar previous sensor element deviates greater than a certain amount (i.e. is worn out or no longer performs well). This may be done using the electro-stimulated erosion mechanism or the enzyme-related erosion mechanism, which are both described above. The enzyme-related erosion mechanism depends on the enzyme in the fluid to degrade the disintegration layer. Therefore, sensor activation is prolonged, so that in a multiple sensor system, when one sensor stops functioning, the next sensor could start functioning without the system needing to be replaced.

In some embodiments, the screening layer 230 may provide biochemical screening. In some alternative embodiments, the screening layer 230 may provide optical screening. In some alternative embodiments, the screening layer 230 may provide both biochemical and optical screening.

In embodiments in which the screening layer 230 provides biochemical screening, a protective coating may be applied to minimize common interferences found in bodily fluids (e.g. serum, interstitial fluid, blood, urine, sweat, tears, etc.). In the case of sensing elements, they may be encapsulated by a screening layer 230 having a positively/negatively charged polymer, a microporous membrane, an anionic/cationic hydrogel, a perflourinated membrane or any combination thereof. When the surface of the FCL is coated with an anionic substance, this may fend off any cationic species from depositing and fouling the components of the FCL.

The use of a negatively charged polymer in the screening layer 230 may limit diffusion of a buffer species (e.g. Cl—, phosphates) but may also limit diffusion of the target analyte. Alternatively, the use of a positively charged polymer in the screening layer 230 may limit diffusion of protein and large molecule interferences (e.g. Ascorbic acid, Uric acid, and Acetaminophen) while promoting diffusion of the target analyte. Limiting access of interferences to the sensor surface may promote increased sensitivity. For example, in glucose sensing applications, limiting access of interferences to a gold surface of a sensing element may promote a kinetically limited glucose oxidation reaction and thereby allow sensing of lower glucose concentrations. Normally, small sized particles are unaffected by the effects of interferences. However, a screening layer may also prevent adhesion of interferences to one or more of the electrodes used in the sensing elements that are polarized.

In embodiments in which the screening layer 230 provides optical screening, the components of the screening layer 230 may be selected so that incident photons with specific wavelength are absorbed and filtered. This may be done by using materials such as optical dye, photosensitive micro/nano-particles or photonic crystals in the screening layer 230.

The sensing element 222 may be the sensor used in the biosensor module described previously. In some embodiments, more than two sensing elements 222 may be implemented in the FCL 190. The sensing element 222 may operate through various mechanisms and modes of action such as, but not limited to, direct oxidation, in-direct oxidation, charge transport, conductivity, and optical stimulation, for example. For example, artificial enzymatic (i.e. enzyme mimicking) sensors display the properties of an enzyme that naturally catalyzes a reaction to detect a target molecule or a target compound. These sensors can perform the direct or in-direct oxidation process. They may be composed of multi-metallic nano-structures which allow enhanced life-span and stability in harsh environment compared to naturally occurring enzymes which denature under varying conditions (e.g. temperature, pH, etc.) as is described in U.S. provisional patent application No. 62/115,886 filed on Feb. 13, 2015, which is hereby incorporated by reference in its entirety. Furthermore, artificial non-enzymatic sensors can detect target analytes for which there are no naturally occurring enzymes that catalyze the reaction to detect the target analyte. These sensors can also perform direct and in-direct oxidation reactions. Due to their highly complex, extremely high surface areas, yet compact and convoluted structures, numerous defect sites may exist for the catalytic reaction of the target.

The optical element 224 may process incident photon or emit photons. Light Emitting Diodes (LEDs) and Organic Light Emitting Diodes (OLEDs) are photon emitting optical elements that may be used as the optical element 224. Liquid Crystal Display (LCD), surface plasmonic resonators, and photonic crystals are candidates may be used as optical elements that do not emit photons, but instead process incident photons that come from outside of the FCL 190. The absorption spectrum of these active optical elements may be electrically controlled to produce desirable wavelengths of photons that are reflected or transmitted upon incidence with the optical element 224. In some embodiments, more than two optical elements 224 may be implemented in the FCL 190.

The substrate 208 hosts or supports all of the functional components of the FCL 190. The substrate 208 may be made from polymer materials such as, but not limited to, polyimide, polyethylene, and polyurethane, for example. The substrate layer 208 provides structural integrity for the FCL 190 during and after the manufacturing process.

In some embodiments, the substrate 208 may be formed by UV polymerization of formulated model contact lens materials such as DMA (Dimethylacrylate), HEMA (2-Hydroxyethl Methacrylate) and Tris (3-[Tris(trimethylsiloxy)silyl]propylmethacrylate) which may provide the substrate 208 with desirable properties such as oxygen permeability and interference minimization.

The IC 220 is an example integrated circuit element which hosts one, many or all of the sensor/optical interface circuits, communication module, and power module. Since it is highly sensitive to water, electric charge and other reactive species, it is insulated by the insulation layer 206 from the rest of the system to protect it from damage.

The insulating layer 206 prevents damage to sensitive electronic components, such as ICs and interconnects. Materials with low water permeability, low oxygen permeability and low charge transport coefficient may be good candidates for the insulation layer 206. For example, parylene based polymers may be used as they are known for their excellent moisture dispelling properties and their excellent biocompatibility.

In the example embodiment of the FCL 190 shown in FIG. 5, the moisture dispelling insulating layer 206 is shown to cover the IC 220 fully, but only cover the antenna 226 partially. In alternative designs, the antenna 226 and interconnects may be covered partially, fully, or not at all. For example, in some cases, such as with the FCL 4 and the FCL 6, part or all of the antenna may change its impedance based on deposition of biomarkers such as salt, proteins or lipids. This information can then be detected by the external device, for the FCL to be cleaned, for example. Sensing elements 222 are not covered by the insulating layer 206 since the sensing elements 222 requires access to surrounding solutions to sense target analytes and the insulating layer 206 prevents this access.

The multipurpose antenna 226 may captures wireless energy from a reader, and transmits signals generated from a communication module in the FCL 190. The antenna 226 may be a piece of highly conductive material that is matched to both the operating frequency of then communication module, and the terminal impedance of the power module used in the FCL. Alternatively, element 226 may represent one of the many interconnects between sensing elements, antennas, integrated circuits, or any other electronic components in the FCL 190.

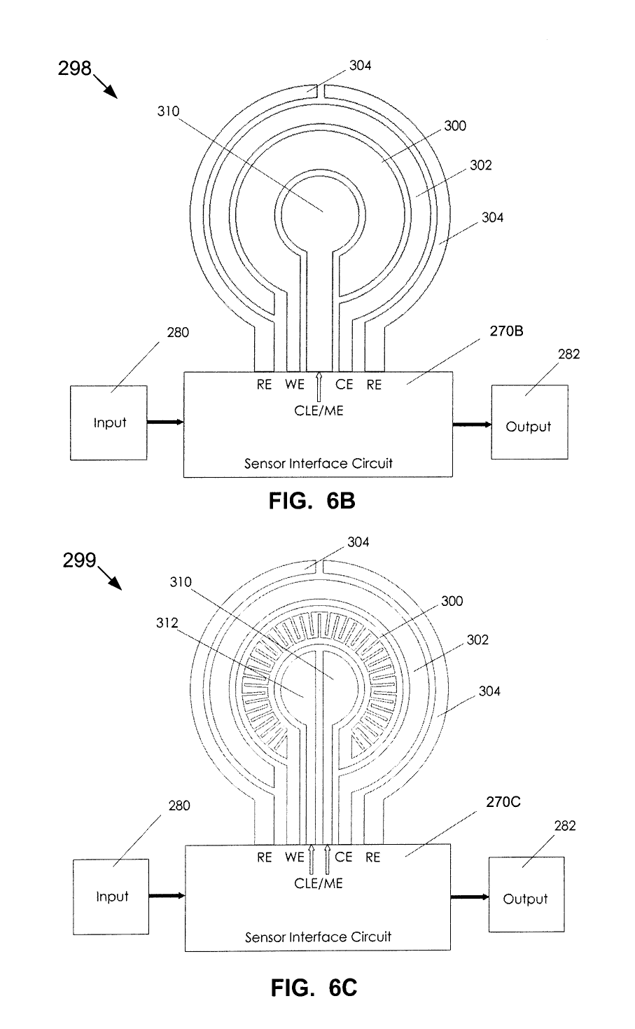

Referring now to FIG. 6A, shown therein is a schematic view of an example embodiment showing a sensor interface circuit 270A and a sensor element 248 along with an input 280 and an output 282 for making measurements during operation. This schematic represents the measurements that may be made by one of the biosensors in FCL 2, FCL 8, or FCL 190 during operation. The sensor element 248 comprises a W.E. 250 and a second electrode 252 that may act as a counter electrode (C.E.) and/or a reference electrode (R.E.). The W.E. has a partial ring or hook shape and is surrounded by the second electrode 252 which also has a partial ring or hook shape that is a larger mirrored version of the W.E. 252 although the widths of the rings making up the W.E. 250 and the second electrode 252 do not have to be the same. In other embodiments, the W.E. 250, the second electrode 252 may have other shapes, besides the one shown in FIG. 6A.

The sensor interface circuit 270A receives the input 280 and converts it to another input form and then perturbs the sensor element 248 using that new input form. Using the new input form, the sensor element 248 will either cause a reaction to occur, or simply confirm the end effects of an already occurred reaction. For example, in oxidation reaction mechanisms involving artificial materials such as glucose for example, the electric potential of the functional layer may be raised to a higher potential to initiate and complete the reaction, yielding an output. However, in the case of a charge transport mechanism where an analyte binds to the sensing element passively e.g. as in a protein binding to antibodies, the input does not initiate the reaction between the analyte and the sensing element; the input only initiates the charge transport mechanism to confirm a lack or a presence of the analyte.

The input 280 may be received from the communication module. The input 280 may be a voltage waveform or a current waveform. For example, the input 280 may be a constant DC voltage, a constant DC current, a step-up DC voltage, a step-up DC current, a sinusoidal AC voltage with a certain radial frequency and amplitude, a sinusoidal AC current with a certain radial frequency and amplitude, a square wave AC voltage or current pulse, or any combination thereof. The sensor interface circuit 270A has the capability to convert from any of the input signal forms to another of these input signal form and then apply the converted input to the sensor element 248.

The sensor interface circuit 270A may then generate the output 282 having a particular form based on the action of the sensing element 248. For example, the output form may be a constant DC voltage, a constant DC current, a sinusoidal AC voltage with a certain radial frequency and amplitude, a sinusoidal AC current with a certain radial frequency and amplitude, a square wave AC voltage or current pulse, or any combination thereof. The output 282 will then be delivered to a system which will interpret it.

Referring now to FIG. 6B, shown therein is a schematic view of an example alternative embodiment showing a sensor interface circuit 270B and a sensor element 298 along with an input 280 and an output 282 for making measurements during operation. In this example embodiment, the sensor element 298 comprises a W.E. 300, a C.E. 302, a R.E. 304 and an electrode 310 that may act as a Cleansing Electrode (CL.E.), a Modulating Electrode (M.E.) or both a CL.E. and a M.E.

In this example embodiment, the electrode 310 that provides the cleansing and/or modulating functions is located at the centre of the electrodes of the sensing element 298. The W.E. 300 extends around the electrode 310 and has a partial ring or hook shape. The C.E. 302 then extends around the W.E. 300 and also has a partial ring or hook shape that is facing the opposite direction as the hook shape of the W.E. 300. The R.E. 304 extends around the C.E. 302 and may have a continuous ring shape or be made of two electrodes that each have complimentary semi-ring shapes.

The electrode layout shown in FIG. 6B where the W.E. 300 is between the CL.E/M.E. 310 and the C.E. 302 may provide better performance since the region between the CL.E./M.E. 310 and the C.E. 302 will be conditioned by the cleansing or modulating properties of the electrode 310. Cleansing will reduce the interference species in the region where the W.E. 300 is situated whereas modulating will produce desirable species where the W.E. 300 is situated. Thus, having the W.E. 300 in this region between the CL.E./M.E. 310 and the C.E. 302 may improve its overall performance, such as potentially increasing one or more of its specificity, stability, lifetime, and sensitivity. Furthermore, having two electrodes function as the R.E. 302 may provide differential information on the local environment of the W.E. 300.

The W.E. 300 provides a unique sensing function for the sensing element 298. The W.E. 300 may be a base non-catalytic electrode onto which a functional layer that provides the unique sensing function is fabricated. The W.E. 300 may provide the ideal physical properties for growth of the functional layer. In addition, the W.E. 300 may assists in transporting the output to the sensor interface circuit 270B. The base electrode portion of the W.E. 300 may be fabricated by using multiple techniques such as, but not limited to, clean room techniques, wet lab techniques, and screen printing techniques, for example.

As previously mentioned, the input 280 is normally supplied to the sensing element 298 such that it causes a perturbation to the W.E. 300. This perturbation is measured as the output 282. For example, artificial enzymatic (i.e. enzyme mimicking) sensors display the properties of an enzyme that naturally catalyzes a reaction to detect a target molecule or a target compound when a constant DC voltage is applied as the input 280. This perturbation allows the sensing element 298 to perform direct or in-direct oxidation or reduction of the target analyte, which in-turn releases electrons from the target analyte. The electrons are funneled through the W.E. 300 to the sensor interface circuit 270B which provides the output 282 to another component for measurement.

The C.E. 302 may be configured to provide a current source or a current sink for the W.E. 300 during sensing operations. This enables continuation of the reaction at the W.E. 300 during analyte sensing. The output at the C.E. 300 may not be measured by the sensor interface circuit 270B. A base metal layer may be deposited in a desired pattern to form the C.E. 302.

The R.E. 304 may be configured to provide a reference level for measurements made at the W.E. 300. In some cases, the R.E. 304 may be one or many electrode(s) (in the example shown in FIG. 6B, two electrodes are used for the R.E. 304.

To form the R.E. 304, a base metal layer may be initially deposited in a desired pattern. This may be followed by activation of this metal layer by addition of a redox active material. This may be achieved, for example, via drop casting, by performing an ion exchange reaction, or by electrochemically plating the active layer. The final step may involve addition of a liquid reference solution as well as designing an interface between the test and the reference solution. In some embodiments, this reference solution may be replaced with a solid-state system which eliminates the associated liquid interface, allowing for more feasible fabrication. For example, the solid state system may be an ion doped membrane (e.g. Agar gel saturated with sodium chloride or Polyvinyl chloride doped with ionic liquid). Furthermore, protective layers such as polyurethane, nafion, or silicon rubber may be added to increase stability of the solid state material.

The electrode 310 may be used as a M.E. and/or a CL.E. A sensing element that uses a 4-electrode system or a 5-electrode system having at least one of a M.E. and a CL.E. may be used to provide additional functions to achieve an improvement in performance over that of 2-electrode systems (e.g. electrodes 300 and 302) or 3-electrode systems (e.g. electrodes 300, 302 and 304).

In some embodiments, a M.E. may be added to 3 or 4 electrode systems to modify local conditions in the micro-environment around the sensing element by creating an abundance of rate-limiting reagents such as oxygen, for example, or by changing the local pH by consumption or production of hydroxide to prime the W.E., for example. However, this temporary micro-environment quickly dissipates as system equilibrium is restored over time, or as all the produced species are consumed. Artificial sensor activity may be enhanced within these micro-environments.

In some embodiments, a CL.E may be added to 3 or 4 electrode systems to eliminate interference species, by using oxidation or decomposition. For example, in some embodiments, the surface of the CL. E. may also include artificial enzymes or reactive species that target specific interference molecules.

In some embodiments, a M.E. and a CL.E. may be added to a 3 electrode sensor system to improve performance. Thus, the addition of the M.E. and the CL.E. serve to further improve sensing performance as compared to when these additional electrodes are used separately.

In some embodiments, such as the sensing element 298, it may also be possible to use one electrode to provide both the modulating and cleansing functions. This may be done in cases where the species that is produced by modulating may also allow the W.E. 300 to selectively bind the target molecule and also repel interference species, thus also providing a cleansing function.

Referring now to FIG. 6C, shown therein is a schematic view of another example alternative embodiment showing a sensor interface circuit 2700 and a sensor element 299 along with an input 280 and an output 282 for making measurements during operation. In this example embodiment, the sensing element 299 comprises an interdigitated electrode design. This, in-turn, alters the fabrication of the active layer drastically. Furthermore, in this example, the M.E. 310 and the CL.E. 312 exists as individual electrodes, that respectively fulfill the modulating and cleansing purposes as outlined for the sensing element 298 in FIG. 6B. It should be noted that the interdigitated design for the working electrode 300 in FIG. 60 may also be used for the working electrodes 300 shown in FIG. 6A or FIG. 6B.

In this example embodiment, the M.E. 310 and the CL.E. 312 are situated at the centre of the electrodes of the sensing element 299. The M.E. 310 and the CL.E. 312 may have similar shapes that face in operate directions. The W.E. 300 extends around the ME. 310 and the CL.E. 312 and is interdigitated with a partial ring or hook shape (in alternative embodiments, interdigitation for the W.E. 300 is not used). The C.E. 302 then extends around the W.E. 300 and also has a partial ring or hook shape that is facing the opposite direction as the hook shape of the W.E. 300. The R.E. 304 extends around the C.E. 302 and may have a continuous ring shape or be made of two electrodes that each have complimentary semi-ring shapes. The interdigitated W.E. 300 may have enhanced performance, due to at least one of increased surface area, highly sensitive sensing structures, highly uniform base currents, and high stability that may all be due to the interdigitation. In some cases, the designs of the W.E. 300 may be designed to have a teeth-like, spiral, or fractal shape which may have at least some of the same benefits as when interdigitation is used.

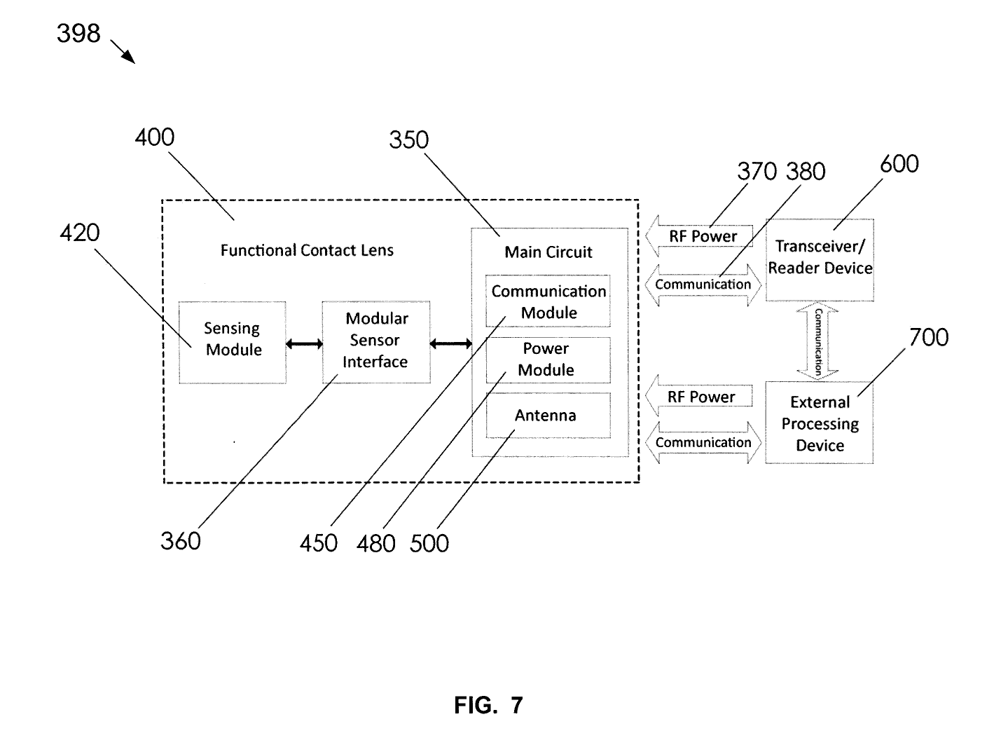

Referring now to FIG. 7, shown therein is a block diagram showing an example embodiment of a telemetry system 398 that comprises an FCL 400, a transceiver-reader device 600 and an external processing device 700. In particular, FIG. 7 shows the energy and information flow between the FCL 400, the transceiver-reader device 600, and the external processing device 700.

In this example embodiment, the FCL 400 comprises a main circuit 350, a modular sensor interface 360 and a sensing module 420. The modular sensor interface 360 couples the main circuit 350 to the sensing module 420. The main circuit 350 may comprise a communication module 450, a power module 480 and an antenna 500. Accordingly, this is an example of a modular design in which modular sensor circuitry, main circuitry and sensor interface circuitry are used to implement the control and sensing functions of the FCL. By keeping the ASICs modular, different sensing/optical modules may be used with the same main circuitry, as long as suitable sensor/optical circuitry are used accordingly.

Radio Frequency (RF) power may be transmitted to the FCL 400 by either the transceiver-reader device 600, or the external processing device 700, or both. The RF power emitted from the transceiver-reader device 600 or the external processing device 700 carries a strong energy component that is sufficient for operations on the FCL 400 and meets FCC safety regulations. The RF power may be received by the antenna 500 and the received RF power may then be conditioned by the power module 480. The voltage generated by the power module 480 may be used to power the main circuit 350 and the modular sensor interface 360. The modular sensor interface 360 in term may apply sufficient voltage/current inputs to condition the sensing module 420 for measurements of certain target analytes in the environment of the FCL 398. Once properly conditioned, the sensing module 420 may facilitate reactions with one or more target biomarkers to generate electrical outputs.

The main circuit 350 and the modular sensor interface 360 may be implemented using Application Specific Integrated Circuits (ASICs).

In some embodiments, the same ASIC may be used to implement both the main circuit 350 and the modular sensor interface 360. In some cases, the ASICs may be separate components.

In some alternative embodiments, multiple interface ASICs may be used to implement the modular sensor interface 360 and these interface ASICs are coupled with the same main circuit ASIC 350.

Detection outputs from the sensing module 420 may be sent by the modular sensor interface 360 to the communication module 450 where they may then be processed. The processed information may be transmitted by the antenna 500 from the FCL 400 to an external device, such as the transceiver-reader device 600 or the external processing device 700. This processed information may also be relayed among devices 600 and 700.

Communication between the FCL 400 and the transceiver-reader device 600 may be bi-directional. Likewise, communication between the FCL 400 and the external processing device 700 may be bi-directional. The FCL 400, the transceiver-reader device 600 and the external processing device 700 may utilize the same radio frequency band as the one used for transmission of RF power 370, or they may allocate another band of the radio spectrum specifically for data transfer 380.

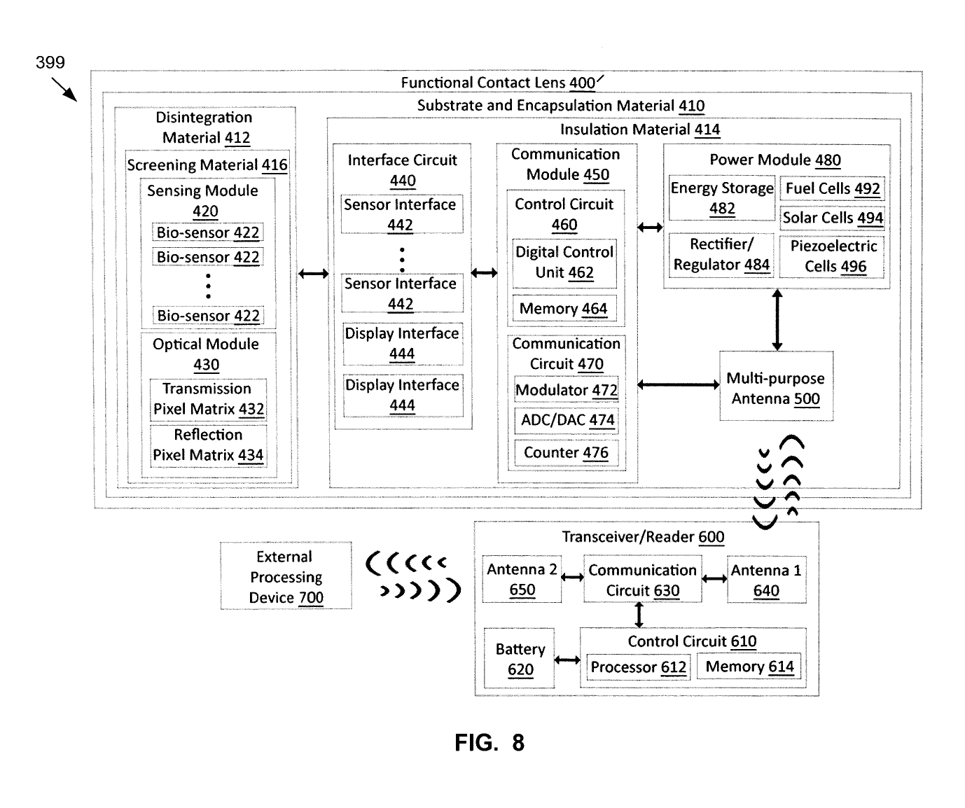

Referring now to FIG. 8, shown therein is a block diagram showing another example embodiment of a telemetry system 399 comprising a FCL 400′, the transceiver/reader device 600, and the external processing device 700.

Within the FCL 400′, all functional components may be encapsulated by the encapsulation material 410 or contact lens materials. Within encapsulation material 410, the sensing module 420 and the optical module 430 may be encapsulated in screening material 416 which may be encapsulated in disintegration material 412. Alternatively, in some embodiments, the screening material 416 may encapsulate the disintegration material 412.

The sensing module 420 may contain one or more sensing elements, or bio-sensors 422. The optical module 430 may contain one or more optical elements such as a transmission pixel matrix 432 or a reflection pixel matrix 434 or a combination thereof. In some embodiments, the optical elements may be light-emitting pixels such as a light emitting diode (LED) or an organic light emitting diode (OLED).

The sensing module 420 and the optical module 430 may be encapsulated in the screening material layer 416 which improves their performance by screening undesirable species or photons. The sensing module 420 and the optical module 430 may be further encapsulated in the disintegration material layer 412 for timed activation of selected sensing elements and/or optical elements for sequential or parallel operations, or a combination of the two.

Encapsulated in the insulation material 414 are generally sensitive electronic components. Such components include one, some, or all of the interface circuit 440, the communication module 450, the power module 480 or the multi-purpose antenna 500. As mentioned in the description related to FIG. 5, the insulation material 414 may include materials with low permeability for reactive species such as water, oxygen, electronic charge, etc., to prevent electronic components from being damaged. In some cases, parts of components 440, 450 480 or 500 may not be insulated. For example, fuel cells 492 from the power module 480 may be exposed to fluid in order to harvest and react with analyte molecules from the fluid to generate energy. Examples of such fuel cells include, but are not limited to, glucose fuel cells, lactate fuel cells, ascorbic acid fuel cells, and uric acid fuel cells.

The interface circuit 440 facilitates transmission of the power supply signals that are applied to the communication module 450 and then routed from the communication module 450 to the sensing module 420 and the optical module 430. The interface circuit 440 also facilitates communication of information collected from the sensing module 420 and/or the optical module 422 back to the communication module 450. Examples of such interface circuits may include, but are not limited to, potential-/galvanic-static circuits for DC sensing elements and/or optical elements, and modulation circuits for AC sensing elements and/or optical elements. Voltage/current multipliers may also be implemented in the interface circuit 440 depending on the strength of certain signals in certain conditions.

The communication module 450 may be used to process signals received by the multipurpose antenna 500, control the interface circuit 440, convert the measured sensor data into digital information, and generate signals for transmission to the transceiver-reader device 600 via the multipurpose antenna 500. Accordingly, the communication module 450 may include a control circuit 460 having a digital control unit 462 and a memory element 464. The communication module 450 may also include a communication circuit 470 having a modulator 472, an Analog to Digital Converter (ADC) and a Digital to Analog Converter (DAC) block 474, and a counter 476. The above functionalities involve collaboration between the control circuit 460 and the communication circuit 470. The control circuit 460 controls the interface circuit 440 and the communication circuit 630.

The communication circuit 470 may convert analog RF signals received by the multipurpose antenna 500 into digital bits for processing by the control circuit 460. The communication circuit 470 may also transmit data from the memory element 464 to the transceiver-reader device 600.

The digital control unit 462 may be a microcontroller for embedded applications that contain a processor core, memory, and input/output terminals for controlling peripheral devices such as the interface circuit 440, and the communication circuit 470. The memory element 464 may be a non-volatile storage to store the data received from the transceiver-reader device 600, and demodulated by the communication circuit 470. The memory element 464 may also be used to store raw measurement data from the interface circuit 440. The memory component may be flash or EEPROM technology.

The modulator 472 may be a network of transistors responsible for converting baseband digital bits stored in the memory element 464 into a passband carrier signal that is suitable for wireless telecommunication via the multipurpose antenna 500.

The ADC/DAC block 474 may be used to convert the analog/digital signals from the interface circuit 440 into digital/analog signals for processing by the control circuit 460.

The counter 476 may be a system clock made from crystal quartz that controls the synchronization of the digital processing across different functions within interface circuit 440, the communication module 450, and the power module 480.

In some embodiments, in order for the communication module 450 to consume low power, analog RFIC technology may be used. This may involve using four major compartments: a potentiostat circuit, a baseband signal conditioning circuit, power electronics, and a radio-frequency signal transmitter with a matching circuit.

The power module 480 may comprise an energy storage unit 482, a rectifier 484, one or more fuel cells 492, one or more solar cells 494 and one or more piezoelectric cells 496. The fuel cells 492, solar cells 494 and piezoelectric cells 496 may be optional in certain embodiments. In some embodiments, the power module 480 may include at least two of the one or more fuel cells 492, the one or more solar cells 494 and the one or more piezoelectric cells 496.

The power module 480 may convert the power captured by the multipurpose antenna 500 via the rectifier 484 and stores the converted power in the power storage unit 482. The use of at least one of one or more fuel cells 492, one or more solar cells 494, and one or more piezoelectric cells 496 may be used to charge the DC power storage unit 482 either independently, or as a combination with the power captured by the multipurpose antenna 500.

The power storage unit 482 may be DC power storage comprising at least one of a super-capacitor and a rechargeable solid element battery. The power storage unit 482 may power the communication module 450 and the interface circuit 440.

The rectifier 484 may be a series of capacitors and diodes that convert a received AC signal from the multipurpose antenna 500 to DC power suitable for energy storage in the energy storage unit 482. Example topologies that may be used for the rectifier 484 include, but are not limited to, a Dickson charge pump voltage doubler, and a combined Switchable full bridge/voltage doubler, for example.

Multiple energy harvesting mechanisms may be used in the power module 480. Once voltage or current is generated, the energy may be stored in the energy storage unit 482, or directly consumed by the microelectronic components such as at least one of the rectifier 484, the interface circuit 440 and the communication module 450.

The fuel cells are energy harvesting units which capture energy through consumption or reaction with fuel molecules. Biofuel cells are able to convert biochemical energy into electrical energy. This is achieved by oxidizing a biofuel (e.g. glucose) that is present in excess supply, thereby, producing electrons at an anode of a given fuel cell. To prevent this reaction from halting due to the shift of equilibrium towards the product side, the products may then be converted into another form and are often reduced at the cathode of the fuel cell. The presence of many biofuels such as glucose, lactate, ascorbate, uric acid has been confirmed within the human tear fluid.

In some embodiments, a 2-electrode setup may be utilized for generating power. One electrode is the biofuel oxidizing anode and the other electrode is the end product reducing cathode and they may both take the form of an enzyme catalyst or an artificial catalyst. The reducing agent may be an oxygen-reducing bio-cathode with a high potential for reducing oxygen at a neutral pH.

Solar cells 494 are photon harvesting units which convert incident photons into electrons. Solar cells may be crystalline, poly-crystalline, or amorphous. Semiconductor materials with dopant are usually used to make solar cells. The semiconductor materials may range from gallium arsenide to graphene, for example. The solar cells 494 may be single junction or multi-junction cells. The solar cells 494 may be part of the main circuit 350, or independently located elsewhere on the FCL 399.

The piezoelectric cells 496 may be energy harvesting units that convert mechanical movements into electrical charges. Example mechanisms of mechanical deformations include electromagnetic, fluidic or mechanical movements. Common materials for such cells may be, but are not limited to, wurtzite materials, metal oxides, ferromagnetic materials, and other semiconducting materials, for example. Piezoelectric potentials are created upon mechanical deformation of the material structure of the piezoelectric cells. Structures such as nano-/micro-pillars, nano-/micro-rods or nanowires, may provide enhanced elastic range for the deformation to take place. One example of such a system is Zinc Oxide nano-pillars. Alternatively, in some embodiments, a micro-motive layer may be disposed at the bottom of the FCL to harvest energy from mechanical friction, bending and stretching of the FCL.

The multipurpose antenna 500 may capture wireless energy from the transceiver-reader device 600 or the external processing device 700, and may transmit signals generated by the communication module 450 to the transceiver-reader device 600 or the external processing device 700. The multipurpose antenna is usually a piece of highly conductive material that is matched to both the operating frequency of the communication module 450, and the terminal impedance of the power module 480.

In some embodiments, one, some or all of the fuel cells 492, solar cells 494, piezoelectric cells 496 and multipurpose antenna 500 may be used as power harvesting mechanisms.

The transceiver-reader device 600 may receive data from the FCL 400. In some cases, the transceiver-reader device 600 may also act as an energy source that wirelessly powers the FCL 400 via the use of antenna 640. The transceiver-reader device 600 may act as either or both the final display unit of received data from the FCL 400, and as an intermediary relay node responsible for forwarding any data received from the FCL 400 to the external processing device 700.

The transceiver-reader device may also be in close proximity to the FCL in order to provide RF power. Accordingly, the transceiver-reader device may be releasably attached to an article that is worn by the user such that the RF transmission pathway between the transceiver-reader device and the FCL is clear of any biological bodies. Thus, transceiver-relay device may be integrated onto eye glasses or it may be in the form of a clip so that the user may attach it to their collar, tie, jacket, shirt or sweater. The relative position of the transceiver-reader device and the FCL may be somewhat constant even when the user moves.

The transceiver-reader device 600 generally comprises a control circuit 610, a communication circuit 630, a power source 620, and an antenna 640 (Antenna 1) for electromagnetic interaction with the FCL 400. The control circuit 610 may comprise a processor 612 and memory 614 to regulate the usage of the power source 620 (which is a battery in this example), and to regulate the operation of the communication circuit 630. The control circuit 610 may also determine the action to take for a particular set of received raw measurement data from the FCL 400.

The processor 612 may be a microcontroller for embedded applications that contain a processor core, memory, and input/output terminals for controlling peripheral devices to limit power consumption from the power source 620, and manage the data from the communication circuit 630. The memory 614 may be a non-volatile storage for the data transmitted from the FCL 400 to the transceiver-reader 600 and demodulated by the communication circuit 630. The memory 614 may use either flash or EEPROM technology.

The power source 620 is typically a DC power source that supplies power to both the control circuit 610 and the communication circuit 630.

The communication circuit 630 may have the capability to transmit and receive at least one of NFC, Bluetooth, and WiFi signals. This may be achieved by either utilizing multiple separated RFICs of the aforementioned technologies, or using an RFIC with this combined capability. The communication circuit 630 outputs digital bits to the control circuit 610 by converting analog signals received from the antennas 640 and 650. The communication circuit 630 may also be able to modulate the digital bits received from the memory 614 for transmission to the FCL 400, and/or the external processing device 700.

The antenna 640 may capture wireless energy from the FCL 400, and may also broadcast signals generated by the communication circuit 630. The antenna 640 may be made using highly conductive material that is matched to both the operating frequency and the terminal impedance of the communication circuit 630.

The antenna 650 may captures wireless energy from the external processing device 700, and may also broadcast signals generated by the communication circuit 630 to the external processing device 700. The antenna 650 may be a highly conductive material that is matched to both the operating frequency and terminal impedance of the communication circuit 460.

The external processing device 700 receives raw sensory data from the transceiver-reader device 600 via a suitable telecommunication standard. If wireless data transmission is used, then the communication is broadcasted from the antenna 650. The external processing device 700 may be implemented using a smartphone, a personal computer, a cloud computing server, and wearable technologies as long as it is capable of analyzing the raw data and displaying it to the user.

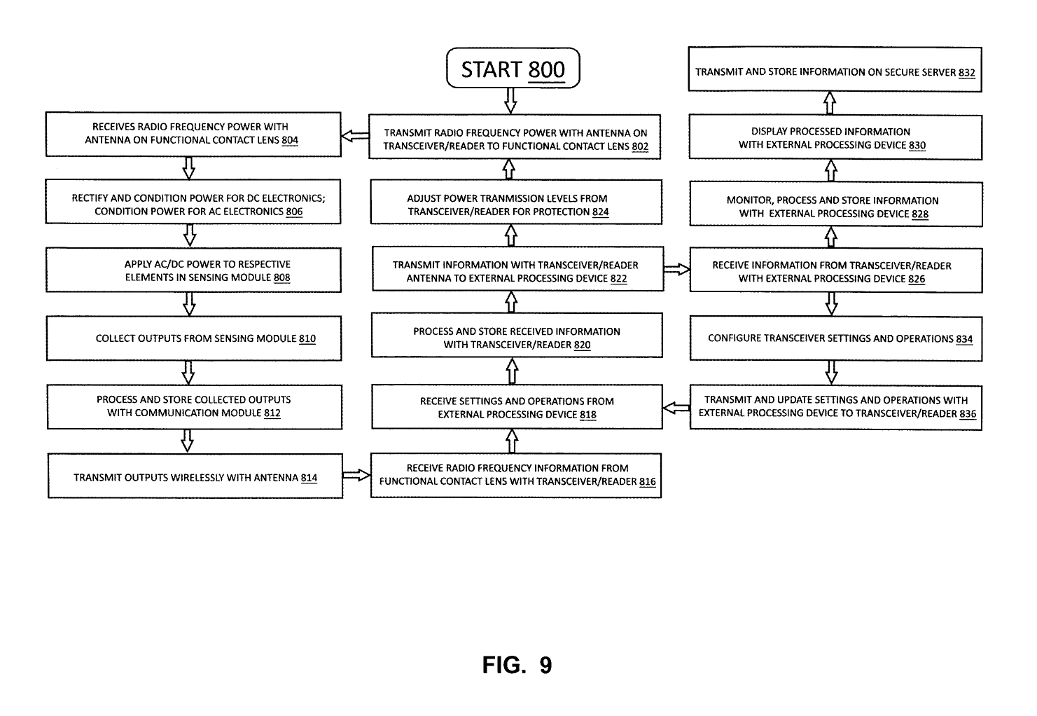

Referring now to FIG. 9, shown therein is an example of flow of power and information among various components of an example FCL including sensing modules, a transceiver-reader device, and an external processing device.

At 802, energy is transmitted from the transceiver-reader device to the FCL at radio frequencies (RF). One example of this wireless powering mechanism is the use of Near Field Communication (NFC) to power Radio Frequency Identification (RFID) devices via inductive coupling mechanisms. Another example is the use of Ultra-High Frequency (UHF) for wireless powering in the far field. The typical frequency range for RF communication and wireless powering lies between about 1 MHz-100 GHz.

At 804, the antenna of the FCL receives the transmitted RF power from the external transceiver-reader device. High power transfer efficiency and coupling efficiency may be achieved by using multi-layer antenna designs for the FCL antenna.

At 806, received RF power may then be rectified and conditioned for the DC electronics in the integrated circuits of the FCL. Otherwise, the RF power may only be conditioned for the AC electronics in the integrated circuits of the FCL. Power quality may be increased by using conditioning elements such as voltage bias, capacitors, and/or voltage regulators. High-pass, low-pass and/or band-pass filters may also be used for conditioning in some embodiments.

At 808, the conditioned power now provides sufficient voltage or current for the active electronic components in the communication module, interface circuits, and/or sensing modules. Under sufficient voltage/current conditions, sensing elements may react with target biomarkers through various mechanisms to produce voltage and current outputs. One example of such a mechanism is the reduction of glucose molecules through redox reactions resulting in the generation of electrons, which may then be collected by the electrodes of the sensing element(s) as measured as outputs at 810.

At 112, sensed outputs from the sensing element(s) may then be processed and temporally stored in the communication module of the FCL. The sensed outputs may be sampled by the ADC 474, and the ADC's outputted digital bits may be allocated by the control circuit 460 to a specific located in a non-volatile memory element.

At 814, information may then be wirelessly transmitted through the multi-purpose antenna on the FCL.

At 816, the external transceiver-reader device receives the information from the FCL using its antenna. The external transceiver-reader device proceeds to process and store this information through its own processor and memory elements as at 820.

If biomarker profile information is sent from the FCL, then this information may then be transmitted to an external processing device with more processing power to further analyze the data, with more storage space to hold the data securely, or to display the data in real-time or in retrospective manners.

In some embodiments, information from the FCL may also be used to adjust at 824 the power transmitted to the FCL from the external transceiver-reader, so that only appropriate amounts of RF power are supplied wirelessly. Examples of such information may include one or more of temperature profiles, voltage levels, current levels, and reactive species levels to determine whether desired reactions are taking place.

At 818, settings and operation information from the external processing device may be received by the external transceiver-reader device, usually with a different antenna at a different frequency compared to the communication between the FCL and the external transceiver-reader device. This setting information may then be processed and stored at 820 and later used at 824 to adjust the power level supplied to the FCL. Meanwhile, this information may be relayed back to the external processing device for connection diagnostic and/or identification purposes at 822. For example, commands may be converted into operations and applied to the transceiver-reader device. Examples of such commands may include the external processing device asking the transceiver-reader device to turn itself off in which case the transceiver-reader device receives and demodulates the command, interprets this command and then turns itself off.

Once the external processing device receives processed biomarker profile from the external transceiver-reader device with its antenna at 826, it may proceed to process this information for display or storage purposes at 828 and 830. Some examples of processing functions include, but are not limited to, raw data processing (e.g. filtering noise, performing a running average, etc.), calibration, profile mapping (e.g. from current/voltage levels to concentration levels), making predictions (e.g. calculation of rate of change, extrapolation of future trend), performing device operations under certain conditions (e.g. functioning/malfunctioning, turned-off, stand-by, etc.), or maintenance actions (e.g. needs cleaning, needs new battery, etc.).

In some embodiments, at 832, the external processing device may then communicate the information to a remote server for secure long term storage. Meanwhile, based on the information received, and analysis generated from data processing, the settings and operations of the external transceiver-reader device may be reconfigured at 834, and transmitted back to the external transceiver-reader device at 836 to be applied at 818. For example, a change in the setting of the transceiver-reader device may be done by writing a different set of register values to its microcontroller. The ability to change the register can dictate how and which sensor is being used for the next measurement.

In an alternative embodiment, the external processing device may also incorporate the functions and operations of the external transceiver-reader device to directly power and communicate with the FCL.

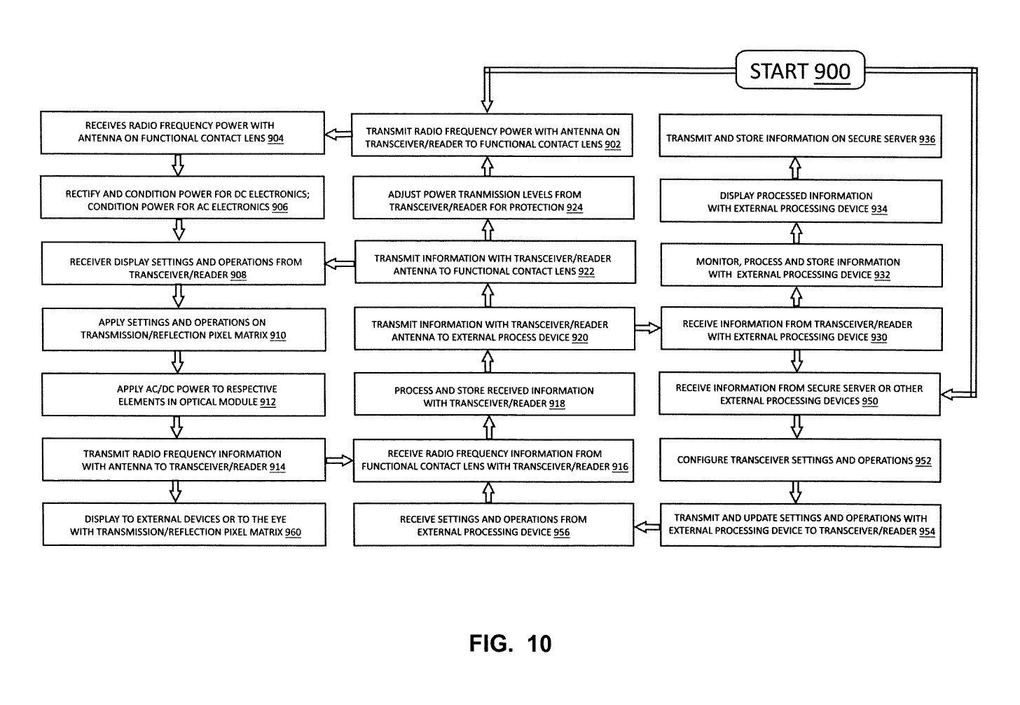

Referring now to FIG. 10, shown therein is an example of the flow of information and power among components of an example FCL including optical modules, an external transceiver-reader device and an external processing device.

At 902, once RF power is wirelessly supplied by the external transceiver-reader device to the FCL, the FCL will receive and process the RF power at acts 904 and 906. Meanwhile, information regarding display settings and operations for the pixel matrices may be received at the FCL and applied at acts 908 and 910. The FCL may then proceed to power the optical modules accordingly at act 912. At 914, information on the performance of the optical module may then be communicated to the external transceiver-reader device through the RF communication antenna. Alternatively, optical feedback may be provided to the transceiver-reader device by the optical module on the FCL at 960. Once feedback is received by the transceiver-reader device via RF or optical means at 916, the external processing device may proceed to process and store such information at 918 and optionally transmit the information to external processing device at 920. For example, the external processing device (or the transceiver-reader device) with antennas and/or photo-detectors (e.g. camera) may be capable of receiver RF or optical feedback from the FCL. This feedback may indicate biomarker levels, actions required (such as the FCL needs cleaning, the FCL needs sanitation, the FCL is worn off), or device performance (such as certain devices being paired, certain devices being functional, certain devices malfunctioning, etc.). The transceiver-reader device may also communicate the processed information back to the FCL, completing the feedback loop at 922. Such information may also be used to adjust power supplied to the FCL for safety and protection purposes at 924.

Meanwhile, FCL display performance information sent by the transceiver-reader device may be received by the external processing device at 930, while the external processing device may constantly receive display information from a remote server or other external processing devices alike at 950. Together, the feedback information and the new information may be combined and processed to configure transceiver settings and operations at 952. For example, configurations may include a series of operation commands for the pixel matrix to display, and for system operations of the transceiver-reader device. In order to provide a reasonable reconfiguration to the transceiver-reader device, information on how well the FCL and the transceiver-device reader are functioning may be taken into consideration, with new commands/operations received by other external processing devices for the FCL to display certain things, for example. The external processing device may then proceed to transmit the processed configuration and operational information to the transceiver-reader device to control the optical modules on the FCL at 956.

At 932 and 934, feedback information from the FCL may also be processed, stored, and displayed on the external processing device. This feedback information may also be communicated to a remote server or other external processing devices alike for secure storage at 936.

In an alternative embodiment, the external processing device may incorporate the functions and operations of the transceiver-reader device so that it may directly power and communicate with the functional contact lens containing the optical modules.

The antennas for each of the embodiments described herein may be made from metals or graphene. The antennas used in the transceiver-reader device and/or the external processing device may have various designs shapes such as patch, annular, coil, or fractal, and may operate in the MHz to GHz range. Micro-antennas made of graphene, may be capable of low frequency RF transmission (e.g. in the MHz range) and have small physical dimensions so as to fit with the design of the overall FCL.

In the various embodiments described in accordance with the teachings herein, minimal RF absorption by the user is desirable when transmitting RF power and information signals. Accordingly, the operational frequencies for RF transmission may be chosen to be suitable for non-invasive biomedical devices.

It should be noted that the various FCLs described in accordance with the teachings herein may be used in one or more applications. For example, the FCLs may be used in one or more of medical diagnostics and monitoring, augmented reality, Intra-ocular imaging and the Internet of Things (IoT).

It should be noted that each of the annular-shaped and wedge-shaped FCLs described in accordance with the teachings herein may be used in FCLs having a single sensor or multiple sensors or having a chip-less design.

It should also be understood that at least some of the elements described herein that are at least partially implemented via software may be written in a high-level procedural language such as object oriented programming or a scripting language. Accordingly, the program code may be written in at least one of C, C⁺⁺, SQL or any other suitable programming language and may comprise modules or classes, as is known to those skilled in object oriented programming. It should also be understood that at least some of the elements of the microcircuitry that are implemented via software may be written in at least one of assembly language, machine language or firmware as needed. In either case, the program code can be stored on a storage media or on a computer readable medium that bears computer usable instructions for one or more processors and is readable by a general or special purpose programmable computing device having at least one processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

Furthermore, the computer readable medium may be provided in various non-transitory forms such as, but not limited to, one or more diskettes, compact disks, tapes, chips, USB keys, magnetic and electronic storage media and external hard drives or in various transitory forms such as, but not limited to, wire-line transmissions, satellite transmissions, internet transmissions or downloads, digital and analog signals, and the like. The computer useable instructions may also be in various forms, including compiled and non-compiled code.

While the applicant's teachings described herein are in conjunction with various embodiments for illustrative purposes, it is not intended that the applicant's teachings be limited to such embodiments. On the contrary, the applicant's teachings described and illustrated herein encompass various alternatives, modifications, and equivalents, without departing from the embodiments described herein, the general scope of which is defined in the appended claims.

### CROSS REFERENCE TO RELATED APPLICATIONS

This application claims the benefit of U.S. Provisional Patent Application No. 61/979,887, filed Apr. 15, 2014, the entire contents of which are hereby incorporated by reference.

### APPENDIX A

### References

Whikehart, D. R., Biochemistry of the Eye, Butterworth-Heinemann, Boston (2003).
Hohenstein-Blaul, N. V. U., Funke, S. & Grus, F. H., Tears as a source of biomarkers for ocular and systemic diseases, Experimental Eye Research 117, 126-137, doi:10.1016/j.exer.2013.07.015 (2013).
Kalsow, C. M., Reindel, W. T., Merchea, M. M., Bateman, K. M. & Barr, J. T., Tear cytokine response to multipurpose solutions for contact lenses, Clinical ophthalmology (Auckland, N. Z.) 7, 1291-1302, doi:10.2147/opth.s44642 (2013).
Lam, S. M. et al., Extensive characterization of human tear fluid collected using different techniques unravels the presence of novel lipid amphiphiles, Journal of Lipid Research 55, 289-298, doi:10.1194/jlr.M044826 (2014).
Longo, U. G. et al., Triglycerides and total serum cholesterol in rotator cuff tears: do they matter?, British Journal of Sports Medicine 44, 948-951, doi:10.1136/bjsm.2008.056440 (2010).
Ohashi, Y., Dogru, M. & Tsubota, K., Laboratory findings in tear fluid analysis, Clinica Chimica Acta, 369, 17-28, doi:10.1016/j.cca.2005.12.035 (2006).
Rassaei, L., Olthuis, W., Tsujimura, S., Sudholter, E. J. R. & van den Berg, A., Lactate biosensors: current status and outlook, Analytical and Bioanalytical Chemistry 406, 123-137, doi:10.1007/s00216-013-7307-1 (2014).
Ronkainen, N. J. & Okon, S. L. Nanomaterial-Based Electrochemical Immunosensors for Clinically Significant Biomarkers. Materials 7, 4669-4709, doi:10.3390/ma7064669 (2014).
Salvisberg, C. et al., Exploring the human tear fluid: Discovery of new biomarkers in multiple sclerosis, Proteomics Clinical Applications, 8, 185-194, doi:10.1002/prca.201300053 (2014).
Schmitt, R. E., Molitor, H. R. & Wu, T., Voltammetric Method for the Determination of Lactic Acid Using a Carbon Paste Electrode Modified with Cobalt Phthalocyanine. International Journal of Electrochemical Science 7, 10835-10841 (2012).
Wei, Y., Gadaria-Rathod, N., Epstein, S. & Asbell, P., Tear Cytokine Profile as a Noninvasive Biomarker of Inflammation for Ocular Surface Diseases: Standard Operating Procedures, Investigative Ophthalmology & Visual Science 54, 8327-8336, doi:10.1167/iovs.13-12132 (2013).
Zhou, L. et al., In-depth analysis of the human tear proteome, Journal of Proteomics 75, 3877-3885, doi:10.1016/j.jprot.2012.04.053 (2012).
Banbury, L. K., 'Stress biomarkers in the tear film, PhD thesis, Southern Cross University, Lismore, N S W, 2009.
Baca, J. T., Finegold, D. N., & Asher, S. A., Tear glucose analysis for the noninvasive detection and monitoring of diabetes mellitus, Ocular Surface 5, 280-293 (2007).
Domschke, A., March, W. F., Kabilan, S., & Lowe, C., Initial clinical testing of a holographic non-invasive contact lens glucose sensor, Diabetes Technology & Therapeutics, 8, 89-93, doi:10.1089/dia.2006.8.89 (2006).
Lane, J. D., Krumholz, D. M., Sack, R. A. & Morris, C., Tear glucose dynamics in diabetes mellitus, Current Eye Research, 31, 895-901, doi:10.1080/02713680600976552 (2006).
March, W. F., Mueller, A., & Herbrechtsmeier, P., Clinical trial of a noninvasive contact lens glucose sensor, Diabetes technology & therapeutics, 6, 782-789, doi:10.1089/dia.2004.6.782 (2004).

1. A functional contact lens for detecting at least one target analyte, comprising:
a substrate for supporting electronic components and providing structural support for the functional contact lens;
at least one sensing element disposed on the substrate for sensing the at least one target analyte and undergoing a physical change representing a sensed signal;
a disintegration layer for covering the at least one sensing element, the disintegration layer being configured to disintegrate during use to allow for operation of the at least one sensing element; and
an antenna disposed on the substrate for transmitting the sensed signal to an external device, the antenna being coupled to the at least one sensing element.

2. The functional contact lens of claim 1, wherein the at least one sensing element undergoes an impedance change when sensing the at least one target analyte, the sensed signal causing a change in a resonance frequency or a change in amplitude of an output signal transmitted by the antenna due to the impedance change of the sensing element.

3. The functional contact lens of claim 2, further comprising an impedance matching element coupled to the antenna for providing impedance matching and compensation of the antenna.

4. The functional contact lens of claim 2, further comprising a second sensing element coupled to the antenna and being configured to undergo an impedance change upon sensing a second target analyte thereby generating another change in a resonance frequency or a change in amplitude of the output signal transmitted by the antenna.

5. The functional contact lens of claim 1, wherein the functional contact lens has a chip-less design.

6. The functional contact lens of claim 1, further comprising:
a main circuit for controlling the operation of the functional contact lens;
a sensing module comprising a plurality of sensing elements for sensing the at least one target analyte during use; and
a modular sensor interface for coupling the main circuit to the sensing module.

7. The functional contact lens of claim 6, wherein the main circuit and the modular sensor interface are implemented using separate Application Specific Integrated Circuits (ASICs) or a common ASIC.

8. The functional contact lens of claim 6, wherein the sensing elements are electrochemical or biochemical sensors.

9. The functional contact lens of claim 8, wherein the biochemical sensors use single strain DNA based detection or antibody based detection.

10. The functional contact lens of claim 6, wherein the main circuit comprises:
the antenna;
a communication module for receiving signals from the external device and transmitting signals to the external device; and
a power module for providing power to components of the functional contact lens requiring power for operation.

11. The functional contact lens of claim 6, wherein the antenna has an annular shape and is disposed along a first annular section of the functional contact lens having a first radius, the sensing elements are disposed along a second annular section of the functional contact lens having a second radius less than the first radius and interconnects are disposed along a third annular section of the functional contact lens between the first and second annular sections to couple the sensing elements with the main circuit, wherein the annular shapes, annular sections and peripheral edge of the functional contact lens are all either circular or oval.

12. The functional contact lens of claim 11, wherein interconnects are disposed between the sensing elements to allow the sensing elements to share one or more electrodes.

13. The functional contact lens of claim 1, wherein the functional contact lens comprises:
a first member having an annular shape and disposed at an outer periphery of the contact lens;
a second member having a disc shape and disposed within and adjacent to the first member, and
the substrate which has a disc shape with a smaller circumference than the second member and is disposed on the second member,
wherein the annular and disc shapes are all either circular or oval.

14. The functional contact lens of claim 13, wherein at least one of the first and second members are made using a gas permeable contact lens material for users who wear the functional contact lens when sleeping.

15. The functional contact lens of claim 13, wherein at least one of the first and second members are made using a soft hydrogel contact lens material having a high water content for users who wear the functional contact lens during daytime.

16. The functional contact lens of claim 1, further comprising:
an insulation layer covering at least a portion of the electronic components disposed on the substrate to provide protection;
a screening layer disposed on the at least one sensing element to provide selective interaction of the at least one sensing element with its environment; and
an encapsulation layer disposed over the insulation layer and the screening layer and portion of the electronic components, the encapsulation layer being configured to comprise selectively allows for diffusion of certain biomolecules towards the substrate.

17. The functional contact lens of claim 16, wherein the at least one sensing element comprises a biochemical sensor and the screening layer is configured to allow for selective transmission of certain biochemicals to the biochemical sensor during use.

18. The functional contact lens of claim 16, further comprising at least one optical element disposed on the substrate and the screening layer covers the at least one optical element and is configured to allow certain target photons at certain wavelengths to pass therethrough to the at least one optical element.

19. The functional contact lens of claim 18, wherein the disintegration layer covers the at least one optical element, the disintegration layer being configured to disintegrate during use to allow for operation of the at least one optical element.

20. The functional contact lens of claim 19, wherein the disintegration layer is configured to disintegrate during use due to electrical stimulation or naturally occurring biochemically active species present in a surrounding fluid.

21. The functional contact lens of claim 19, further comprising multiple similar sensing elements and sensing lifetime is prolonged by configuring the disintegration layer to disintegrate over a subsequent sensing element after a previous operational sensing element stops functioning or is performing poorly.

22. The functional contact lens of claim 19, wherein the disintegration layer is configured for timed activation of at least one of selected sensing elements and selected optical elements for sequential, parallel or sequential and parallel operation thereof.

23. The functional contact lens of claim 18, wherein the at least one optical element comprises at least one of reflection pixel matrices, transmission pixel matrices, Light Emitting Diodes (LEDs) and Organic Light Emitting Diodes (OLEDs), Liquid Crystal Display (LCD), surface plasmonic resonators, and photonic crystals.

24. The functional contact lens of claim 18, wherein an absorption spectrum of the at least one optical element is electrically controlled to produce desirable wavelengths of photons that are reflected or transmitted upon incidence with the at least one optical element.

25. The functional contact lens of claim 10, wherein the power module comprises:
a rectifier for converting harvested energy conditioning the stored energy to provide power;
an energy storage unit for storing converted harvested energy for use by electronic components of the functional contact lens; and
at least one energy harvesting element comprising at least one of one or more fuel cells, one or more solar cells and one or more piezoelectric cells.

26. The functional contact lens of claim 25, wherein the one or more piezoelectric cells comprise one of micro-pillars and nano-pillars that create a piezoelectric potential upon mechanical deformation.

27. The functional contact lens of claim 1, wherein the at least one sensing element is coupled with a sensor interface for receiving an input signal and providing an output signal, the at least one sensing element comprising:
a working electrode having an annular shape; and
a second electrode having an annular shape with a larger radius that surrounds a majority of the working electrode, the second electrode being configured to operate as a counter electrode or a reference electrode.

28. The functional contact lens of claim 1, wherein the at least one sensing element is coupled with a sensor interface for receiving an input signal and providing an output signal, the at least one sensing element comprising:
a working electrode having an annular shape;
a counter electrode having an annular shape that surrounds a majority of the working electrode; and
a reference electrode having an annular shape that surrounds the counter electrode.

29. The functional contact lens of claim 28, wherein the reference electrode comprises two electrodes having semi-annual shapes disposed on either side of the counter electrode.

30. The functional contact lens of claim 28, wherein the at least one sensing element comprises a fourth electrode disposed within the annular shape of the working electrode, the fourth electrode being configured to provide at least one of a modulating function and a cleansing function of a microenvironment of the working electrode.

31. The functional contact lens of claim 28, wherein the at least one sensing element comprises a modulating electrode and a cleansing electrode that are both disposed within the annular shape of the working electrode and being configured to provide a modulating function and a cleansing function, respectively, of a microenvironment of the working electrode.

32. The functional contact lens of claim 27, wherein the working electrode is interdigitated.

33. The functional contact lens of claim 27, wherein the input signal and the output signal are at least one of a constant DC voltage, a constant DC current, a step-up DC voltage, a step-up DC current, a sinusoidal AC voltage with a certain radial frequency and amplitude, a sinusoidal AC current with a certain radial frequency and amplitude, a square wave AC voltage, and a square wave AC current pulse.

34. The functional contact lens of claim 1, wherein the at least one target analyte comprises at least one of acids, ions, carbonhydrate, proteins, enzymes, lipids, antigens, hormones, nucleic acids, small molecules, medications and recreational drugs.

35. The contact lens of claim 1, wherein the substrate has an annular or wedge shape.

36. A system for monitoring a person's health, wherein the system comprises:
a Functional Contact Lens (FCL) that monitors at least one condition for the person, the FCL comprising at least one sensing element to monitor the at least one condition and a disintegration layer that covers the at least one sensing element and is configured to disintegrate during use to allow for operation of the at least one sensing element;
a transceiver-reader device that communicates with the FCL; and
an external processing device,
wherein the transceiver-reader device acts as a relay device in sending signals between the FCL and the external processing device.

37. The system of claim 36, wherein the FCL further comprises:
a substrate for supporting electronic components and providing structural support for the functional contact lens; and
an antenna disposed on the substrate for transmitting the sensed signal to an external device, the antenna being coupled to the at least one sensing element,
wherein the at least one sensing element is disposed on the substrate for sensing the at least one target analyte and undergoing a physical change representing a sensed signal.

38. (canceled)

39. The system of claim 36, wherein communication between the FCL and the transceiver-reader device is bi-directional.

40. The system of claim 36, wherein the FCL directly communicates with the external reader device and the communication is bi-directional.

41. The system of claim 36, wherein at least one of the transceiver-reader device and the external reader device are configured to provide RF power signals to the FCL to wirelessly power the FCL.

42. The system of claim 36, wherein at least one of the transceiver-reader device and the external processing device are configured to perform at least one of processing and displaying received data from the FCL.
